(19) United States
(12) Patent Application Publication
(10) Pub. No.: US 2009/0285803 A1
(43) Pub. Date: Nov. 19, 2009

(54) ANTI-PirB ANTIBODIES

(76) Inventors: Jasvinder Atwal, San Carlos, CA (US); Marc Tessier-Lavigne, Woodside, CA (US); Yan Wu, Foster City, CA (US)

Correspondence Address: Goodwin Procter LLP;Attn: Patent Administrator, 135 Commonwealth Drive, Menlo Park, CA, 94025-1105, US

(21) Appl. No.: 12/465,169

(22) Filed: May 13, 2009

Related U.S. Application Data
(60) Provisional application No. 61/052,949, filed on May 13, 2008.

(51) Int. Cl. A61K 39/395; C07K 16/00; C12N 15/13; C12N 15/85; C12N 1/00; C12N 5/16; C12P 21/02; C07K 16/42; A61P 25/28

(52) U.S. Cl. 424/131.1; 530/387.3; 536/23.53; 435/320.1; 435/243; 435/327; 435/69.6; 530/387.2; 424/133.1

(57) ABSTRACT

The present invention relates generally to neural development and neurological disorders. The invention specifically concerns identification of novel modulators of the myelin-associated inhibitory system and various uses of the modulators so identified.

### CROSS REFERENCE TO RELATED APPLICATIONS

The present application claims priority under 35 USC §119 to U.S. Provisional Application No. 61/052,949, filed May 13, 2008, and claims priority under 35 USC §120 to U.S. application Ser. No. 12/208,883, filed Sep. 11, 2008 and U.S. application Ser. No. 12/316,130, filed Dec. 9, 2008, all of which are fully incorporated herein by reference.

### FIELD OF THE INVENTION

The present invention relates generally to neural development and neurological disorders. The invention specifically concerns identification of novel modulators of the myelin-associated inhibitory system and various uses of the modulators so identified.

### BACKGROUND OF THE INVENTION

### Myelin and Myelin-Associated Proteins

It is known that axons of the adult mammalian CNS neurons have very limited capacity to regenerate following injury, whereas axons in the peripheral nervous system (PNS) regenerate rapidly. It has been known that CNS neuron's limited capacity to regenerate is in part to an intrinsic property of CNS axons, but also due to an impermissible environment. The CNS myelin, while it is not the only source of inhibitory cues for neurite growth, contains numerous inhibitory molecules that actively block axonal growth and therefore constitutes a significant barrier to regeneration. Three of such myelin-associated proteins (MAPs) have been identified: Nogo (also known as NogoA) is a member of the Reticulon family of proteins having two transmembrane domains; myelin-associated glycoprotein (MAG) is a transmembrane protein of the Ig superfamily; and OMgp is a leucine rich repeat (LRR) protein with a glycosylphosphatidylinositol (GPI) anchor. Chen et al., Nature 403:434-39 (2000); GrandPre et al., Nature 417:439-444 (2000); Prinjha et al., Nature 403:383-384 (2000); McKerracher et al, Neuron 13:805-11 (1994); Wang et al, Nature 417:941-4 (20020: Kottis et al J. Neurochem 82:1566-9 (2002). A portion of NogoA, Nogo66, has been described as a 66-amino acid extracellular polypeptide that is found in all three isoforms of Nogo.

Despite their structural differences, all three inhibitory proteins (including Nogo66) have been shown to bind the same GPI-anchored receptor, called Nogo receptor (NgR; also known as Nogo Receptor-1 or NgR1), and it has been proposed that NgR might be required for mediating the inhibitory actions of Nogo, MAG and OMgp. Fournier et al., Nature 409:341-346 (2001). Two NgR1 homologs (NgR2 and NgR3) have also been identified. US 2005/0048520 A1 (Strittmatter et al.), published Mar. 3, 2005. Given that NgR is a GPI-anchored cell surface protein, it is unlikely to be a direct signal transductor (Zheng et al., Proc. Natl. Acad. Sci. USA 102:1205-1210 (2005)). Others have suggested that the neurotrophin receptor p75^{NTR} acts as a co-receptor for NgR and provides the signal-transducing moiety in a receptor complex (Wang et al., Nature 420:74-78 (2002); Wong et al., Nat. Neurosci. 5:1302-1308 (2002)).

### PirB and Human Orthologs

The major histocompatibility complex (MHC) class I was originally identified as a region encoding a family of molecules that are important for the immune system. Recent evidences have indicated that MHC class I molecules have additional functions in the development and adult CNS. Boulanger and Shatz, Nature Rev Neurosci. 5:521-531 (2004); US 2003/0170690 (Shatz and Syken), published Sep. 11, 2003. Many of the MHC class I members and their binding partners are found to be expressed in CNS neurons. Recent genetic and molecular studies have focused on the physiological functions of CNS MHC class I, and the initial results suggested that MHC class I molecules might be involved in activity-dependent synaptic plasticity, a process during which the strength of existing synaptic connections increases or decreases in response to neuronal activity, followed by long term structural alterations to circuits. Moreover, the MHC class I encoding region has also been genetically linked to a wide variety of disorders with neurological symptoms, and abnormal functions of MHC class I molecules are thought to contribute to the disruption of normal brain development and plasticity.

One of the known MHC class I receptors in the immune setting is PirB, a murine polypeptide that was first described by Kubagawa et al., Proc. Nat. Acad. Sci. USA 94:5261-6 (1997). Mouse PirB has several human orthologs, which are members of the leukocyte immunoglobulin-like receptor, subfamily B (LILRB), and are also referred to as “immunoglobulin-like transcripts” (ILTs) The human orthologs show significant homology to the murine sequence, from highest to lowest in the following order: LILRB3/ILT5, LILRB1/ILT2, LILRB5/ILT3, LILRB2/ILT4, and, just as PirB, are all inhibitory receptors. LILRB3/ILT5 (NP_{—}006855) and LILRB1/ILT2 (NP_{—}006660) were first described by Samaridis and Colonna, Eur. J. Immunol. 27(3):660-665 (1997) LILRB5/ILT3 (NP_{—}006831) has been identified by Borges et al., J. Immunol. 159(11):5192-5196 (1997). LILRB2/ILT4 (also known as MIR10), was identified by Colonna et al., J. Exp. Med. 186:1809-18 (1997). PirB and its human orthologs show a great degree of structural variability. The sequences of various alternatively spliced forms are available from EMBL/GenBank, including, for example, the following accession numbers for human ILT4 cDNA: ILT4-c11 AF009634; ILT4-c117 AF11566; ILT4-c126 AF11565. As noted above, the PirB/LILRB polypeptides are MHC Class I (MHCI) inhibitory receptors, and are known for their role in regulating immune cell activation (Kubagawa et al., supra; Hayami et al., J. Biol. Chem. 272:7320 (1997); Takai et al., Immunology 115:433 (2005); Takai et al., Immunol. Rev. 181:215 (2001); Nakamura et al. Nat. Immunol. 5:623 (2004); Liang et al., Eur. J. Immunol. 32:2418 (2002)).

A recent study by Syken et al. (Science 313:1795-800 (2006)) reported that PirB is expressed in subsets of neurons throughout the brain. In mutant mice lacking functional PirB, cortical ocular dominance (OD) plasticity is significantly enhanced at all ages, suggesting PirB's function in restricting activity-dependent plasticity in visual cortex.

### SUMMARY OF THE INVENTION

The present invention is based, at least in part, on the finding that interfering with PirB activity using function-blocking anti-PirB antibodies helps rescuing neurite outgrowth inhibition by Nogo66 and myelin, and that blocking PirB and NgR activities concurrently leads to a near-complete release from myelin inhibition.

In one aspect, the invention concerns an isolated anti-PirB/LILRB antibody that binds essentially to the same epitope on human PirB (LILRB) as an antibody selected from the group consisting of YW259.2, YW259.9 and YW259.12.

In another aspect, the invention concerns an isolated anti-PirB/LILRB antibody that competes for binding to human PirB (LILRB) with an antibody selected from the group consisting of YW259.2, YW259.9 and YW259.12.

In yet another aspect, the invention concerns an isolated anti-PirB/LILRB antibody that comprises one, two, or three hypervariable region sequences from a heavy chain selected from the group consisting of: YW259.2 heavy chain (SEQ ID NO: 4 or 11), YW259.9 heavy chain (SEQ ID NO: 5 or 12), and YW259.12 heavy chain (SEQ ID NO: 6 or 13).

In an embodiment, the antibody comprises all hypervariable region sequences of the YW259.2 antibody heavy chain (SEQ ID NO: 4 or 11).

In another embodiment, the antibody comprises all hypervariable region sequences of the YW259.9 antibody heavy chain (SEQ ID NO: 5 or 12).

In yet another embodiment, the antibody comprises all hypervariable region sequences of the YW259.12 antibody heavy chain (SEQ ID NO: 6 or 13).

In a further embodiment, the antibody comprises a light chain.

In a still further embodiment, the antibody comprises one, two or three hypervariable region sequences of a light chain from the polypeptide sequence of SEQ ID NO: 7.

In yet another embodiment, the antibody comprises all hypervariable region sequences of a light chain comprising the polypeptide sequence of SEQ ID NO: 7 or 15.

In a specific embodiment, the antibody comprises both a heavy and a light chain, where the heavy chain comprises one, two, or three hypervariable region sequences from a heavy chain selected from the group consisting of: YW259.2 heavy chain (SEQ ID NO: 4 or 11), YW259.9 heavy chain (SEQ ID NO: 5 or 12), and YW259.12 heavy chain (SEQ ID NO: 6 or 13), and/or the light chain comprises one, two or three hypervariable region sequences of a light chain from the polypeptide sequence of SEQ ID NO: 7 or 15.

In a further embodiment, the antibody is selected from the group consisting of antibodies YW259.2, YW259.9, and YW259.12.

In a further aspect, the invention concerns an isolated anti-PirB antibody wherein the full-length IgG form of the antibody specifically binds human PirB with a binding affinity of 5 nM or better, or 1 nM or better.

In an embodiment, the antibody promotes axonal regeneration, such as regeneration of CNS neurons.

In another embodiment, the antibody, at least partially, rescues neurite outgrowth inhibition by Nogo66 and myelin.

In all aspects, the antibody preferably is a monoclonal antibody, which may, for example, be a chimeric antibody, a humanized antibody, an affinity matured antibody, a human antibody, or a bispecific antibody, an antibody fragment or an immunoconjugate.

In a further aspect, the invention concerns a polynucleotide encoding an anti-PirB/LILRB antibody herein.

In other aspects, the invention concerns vectors and host cells comprising a polynucleotide encoding an antibody (including coding sequences of one or more antibody chains) herein. The host cells include prokaryotic, eukaryotic and mammalian hosts.

In a further aspect, the invention concerns a method for making an anti-PirB/LILRB antibody, comprising (a) expressing a vector comprising nucleic acid encoding the antibody in a suitable host cell, and (b) recovering the antibody.

In a still further aspect, the invention concerns a composition comprising an anti-PirB/LILRB antibody herein, and a pharmaceutically acceptable excipient. Optionally, the composition comprises a second medicament, wherein the anti-PirB/LILRB antibody is a first medicament. The second medicament may, for example, be a NgR inhibitor, such as an anti-NgR antibody.

In a different aspect, the invention concerns a kit comprising an anti-PirB/LILRB antibody herein.

In another aspect, the invention concerns a method for promoting axon regeneration comprising administering to a subject in need an effective amount of an anti-PirB/LILRB antibody herein. Preferably, the subject is a human patient.

In embodiments, the treatment method herein enhances survival or neurons and/or induces the outgrowth of neurons

In yet another aspect, the invention concerns a method of treating a neurodegenerative disease, comprising administering to a subject in need an effective amount of an anti-PirB/LILRB antibody herein. The neurodegenerative disease may, for example, be characterized by physical damage to the central nervous system, and includes, without limitation, brain damage associated with stroke.

In a particular embodiment, the neurodegenerative disease is selected from the group consisting of trigeminal neuralgia, glossopharyngeal neuralgia, Bell's Palsy, myasthenia gravis, muscular dystrophy, amyotrophic lateral sclerosis (ALS), multiple sclerosis (MS), progressive muscular atrophy, progressive bulbar inherited muscular atrophy, peripheral nerve damage caused by physical injury (e.g., burns, wounds) or disease states such as diabetes, kidney dysfunction or by the toxic effects of chemotherapeutics used to treat cancer and AIDS, herniated, ruptured or prolapsed invertebrate disk syndromes, cervical spondylosis, plexus disorders, thoracic outlet destruction syndromes, peripheral neuropathies such as those caused by lead, dapsone, ticks, prophyria, Gullain-Barre syndrome, Alzheimer's disease, Huntington's Disease, and Parkinson's disease.

The invention further concerns an anti-idiotype antibody that specifically binds an anti-PirB antibody herein.

### BRIEF DESCRIPTION OF THE DRAWINGS

FIGS. 1A and 1B show the mouse PirB sequence (SEQ ID NO: 1) and the human LILRB2 sequence (SEQ ID NO: 2).

FIGS. 2A and 2B. Blocking PirB reverses inhibition of CGN outgrowth on AP-Nogo66 or myelin. Dissociated mouse P7 CGN were plated on PDL/laminin (control), AP-Nogo66, or myelin to test inhibition by these substrates. (A) Representative photomicrographs, (B) a graph measuring average neurite length (±SE) from one representative experiment. Neurons grown on PDL/laminin, AP-Nogo66, or myelin were cultured in the presence or absence of function-blocking antibodies to PirB (aPB1; 50 μg/ml). aPB1 significantly reduced inhibition by either substrate. (*p<0.01; Scale bars, 50 μm)

FIGS. 3A-3D. Blocking PirB reverses inhibition of CGN outgrowth on AP-Nogo66 or myelin. Dissociated mouse P7 CGN were plated on PDL/laminin (control), AP-Nogo66, or myelin to test inhibition by these substrates. Representative photomicrographs are shown in FIGS. 3A and 3C and a graph measuring average neurite length (±SE) from one representative experiment is shown in FIGS. 3B and 3D. Neurons grown on PDL/laminin, AP-Nogo66, or myelin were cultured in the presence or absence of function-blocking antibodies to PirB (aPB1; 50 μg/ml). aPB1 significantly reduced inhibition by either substrate. (*p<0.01; Scale bars, 50 μm)

FIGS. 4A and 4B. Both PirB and NgR are required to mediate growth cone collapse by myelin inhibitors. Growth cones of postnatal DRG axons were treated with medium alone (control), myelin (3 μg/ml), or AP-Nogo66 (100 nM) for 30 minutes to stimulate collapse, and stained with Rhodamine-phalloidin to visualize growth cones. (A) Representative photomicrographs, (B) a graph measuring percent growth cone collapse (±SEM) from cumulative experiments. Either genetic loss of NgR or inhibition of PirB by anti-PirB treatment was sufficient alone to prevent the growth cone collapsing activity of myelin or AP-Nogo66. Inhibition of both pathways also fully blocked collapse. (Scale bars, 50 μm)

FIGS. 5A-5D. Blocking PirB partially disinhibits neurite outgrowth in DRG neurons and on the substrate MAG. Representative photomicrographs are shown in FIGS. 5A and 5C; graphs showing the average neurite length (±SE) from one representative experiment are shown in FIGS. 5B and 5D. (A) and (B) Dissociated P10 DRG neurons were plated on PDL/laminin, AP-Nogo66, or myelin in the presence or absence of anti-PirB. There was a significant reduction in inhibition by AP-Nogo66 and myelin by aPB1. (C) and (D) Dissociated P7 CGN cultures were plated on PDL/laminin or MAG-Fc, with or without aPB1. Antibodies to PirB reduced the inhibition of neurite outgrowth by MAG-Fc. (* p<0.01; Scale bars, 200 μm A, B; 50 μm C).

FIG. 6. DNA sequence of anti-PirB antibody YW259.2 heavy chain (SEQ ID NO: 8).

FIG. 7. DNA sequence of anti-PirB antibody YW259.9 heavy chain (SEQ ID NO: 9).

FIG. 8 DNA sequence of anti-PirB antibody YW259.12 heavy chain (SEQ ID NO: 3).

FIG. 9 Protein sequence of anti-PirB antibody YW259.2 heavy chain (SEQ ID NO: 4).

FIG. 10. Protein sequence of anti-PirB antibody YW259.9 heavy chain (SEQ ID NO: 5).

FIG. 11. Protein sequence of anti-PirB antibody YW259.12 heavy chain (SEQ ID NO: 6).

FIG. 12. Protein sequence of the light chain of all YW259 antibodies (SEQ ID NO: 7).

FIG. 13. Ability of anti-PirB antibody YW259.2(IgG) to inhibit the activity of His-tagged mouse PirB.

FIG. 14. Ability of anti-PirB antibody YW259.9 (IgG) to inhibit the activity of His-tagged mouse PirB.

FIG. 15. Ability of anti-PirB antibody YW259.12 (IgG) to inhibit the activity of His-tagged mouse PirB.

FIG. 16. Relative AP-Nogo66 binding of a panel of anti-PirB antibodies, including YW259.2, YW259.9, and YW259.12.

FIGS. 17A-17C. Alignment of heavy chain sequences of anti-PirB antibodies YW259.2 (SEQ ID NO: 11); YW259.9 (SEQ ID NO: 12) and YW259.12 (SEQ ID NO: 13). The CDR H1, CDR H2 and CDR H3 sequences are boxed, along with the CDR H domains according to Kabat, Chothia and the contact CDR H domains. Hum III is disclosed as SEQ ID NO:10.

FIGS. 18A-18C. Alignment of the light chain sequences of anti-PirB antibodies YW259.2 (SEQ ID NO: 15); YW259.9 (SEQ ID NO:15) and YW259.12 (SEQ ID NO: 15), and HuκI (SEQ ID NO: 14). The CDR L1, CDR L2 and CDR L3 sequences are boxed, along with the CDR L domains according to Kabat, Chothia and the contact CDR L domains. Hum III is disclosed as SEQ ID NO: 10.

FIG. 19. C1QTNF5 (CTRP5; NP_{—}05646) inhibits neurite outgrowth of dorsal root ganglion neurons, and this inhibition is reduced when PirB is blocked by PirB function-blocking antibody YW259.2.

### DETAILED DESCRIPTION OF THE INVENTION

### A. Definitions

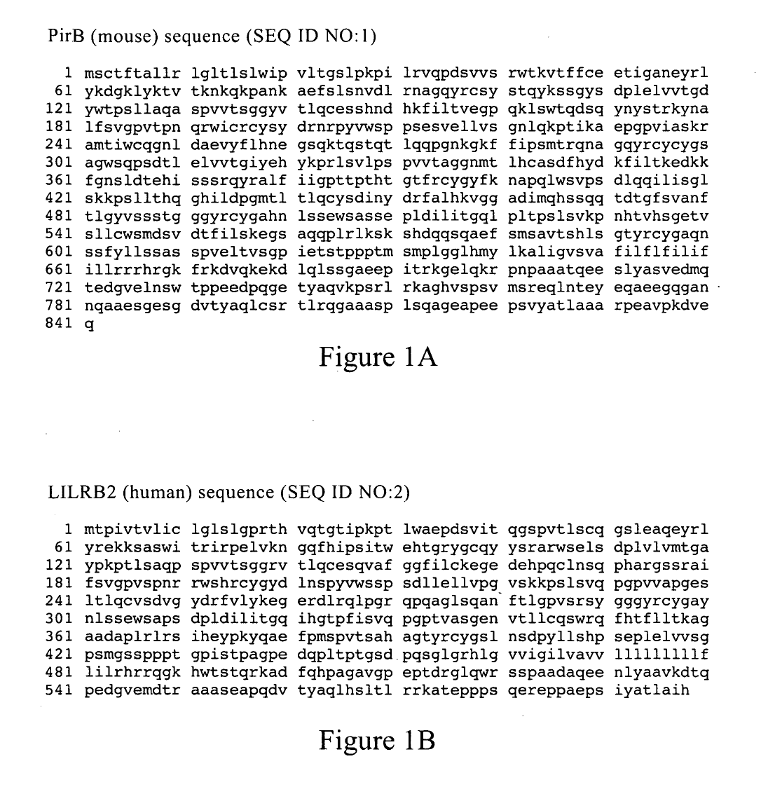

The terms “paired-immunoglobulin-like receptor B” and “PirB” are used herein interchangeably, and refer to a native-sequence, 841-amino acid mouse inhibitory protein of SEQ ID NO: 1 (FIG. 1) (NP_{—}035225), and its native-sequence homologues in rat and other non-human mammals, including all naturally occurring variants, such as alternatively spliced and allelic variants and isoforms, as well as soluble forms thereof. For further details see, Kubagawa et al., Proc Natl Acad Sci USA 94, 5261 (1997).

The terms “LILRB,” “ILT” and “MIR,” are used herein interchangeably, and refer to all members of the human “leukocyte immunoglobulin-like receptor, subfamily B”, including all naturally occurring variants, such as alternatively spliced and allelic variants and isoforms, as well as soluble forms thereof. Individual members within this B-type sub-family of LILR receptors are designated by numbers following the acronym, such as, for example, LILRB3/ILT5, LILRB1/ILT2, LILRB5/ILT3, and ILIRB2/ILT4, where a reference to any individual member, unless otherwise noted, also includes reference to all naturally occurring variants, such as alternatively spliced and allelic variants and isoforms, as well as soluble forms thereof. Thus, for example, “LILRB2,” “LIR2,” and “MIR10” are used herein interchangeably and refer to the 598-amino acid polypeptide of SEQ ID NO:2 (FIG. 1) (NP_{—}005865), and its naturally occurring variants, such as alternatively spliced and allelic variants and isoforms, as well as soluble forms thereof. For further details, see Martin et al., Trends Immunol. 23, 81 (2002).

The term “PirB/LILRB” is used herein to jointly refer to the corresponding mouse and human proteins and native sequence homologues in other non-human mammals, including all naturally occurring variants, such as alternatively spliced and allelic variants and isoforms, as well as soluble forms thereof.

The term “myelin-associated protein” is used in the broadest sense and includes all proteins present in CNS myelin that inhibit neuronal regeneration, including Nogo, MAG and OMgp.

“Isolated,” when used to describe the various proteins disclosed herein, means protein that has been identified and separated and/or recovered from a component of its natural environment. Contaminant components of its natural environment are materials that would typically interfere with diagnostic or therapeutic uses for the protein, and may include enzymes, hormones, and other proteinaceous or non-proteinaceous solutes. In preferred embodiments, the protein will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain, or (3) to homogeneity by mass spectroscopic or peptide mapping techniques. Isolated protein includes protein in situ within recombinant cells, since at least one component of the natural environment of the protein in question will not be present. Ordinarily, however, isolated protein will be prepared by at least one purification step.

An “isolated” nucleic acid molecule is a nucleic acid molecule that is identified and separated from at least one contaminant nucleic acid molecule with which it is ordinarily associated in the natural source of the nucleic acid in question. An isolated nucleic acid molecule is other than in the form or setting in which it is found in nature. Isolated nucleic acid molecules therefore are distinguished from the nucleic acid molecules as they exist in natural cells. However, an isolated nucleic acid molecule includes nucleic acid molecules contained in cells that ordinarily express such nucleic acid where, for example, the nucleic acid molecule is in a chromosomal location different from that of natural cells.

As used herein, the term “PirB/LILRB antagonist” is used to refer to an agent capable of blocking, neutralizing, inhibiting, abrogating, reducing or interfering with PirB/LILRB activities. Particularly, the PirB/LILRB antagonist interferes with myelin associated inhibitory activities, thereby enhancing neurite outgrowth, and/or promoting neuronal growth, repair and/or regeneration. In a preferred embodiment, the PirB/LILRB antagonist inhibits the binding of PirB/LILRB to Nogo66 and/or MAG and/or OMgp by binding to PirB/LILRB. PirB/LILRB antagonists include, for example, antibodies to PirB/LILRB and antigen binding fragments thereof, truncated or soluble fragments of PirB/LILRB, Nogo 66, MAG or OMgp that are capable of sequestering the binding between PirB/LILRB and Nogo 66, or between PirB/LILRB and MAG, or between PirB/LILRB and OMgp and small molecule inhibitors of the PirB/LILRB related inhibitory pathway. PirB/LILRB antagonists also include short-interfering RNA (siRNA) molecules capable of inhibiting or reducing the expression of PirB/LILRB mRNA. A preferred PirB/LILRB antagonist is an anti-PirB/LILRB antibody.

The term “antibody” herein is used in the broadest sense and specifically covers intact antibodies, monoclonal antibodies, polyclonal antibodies, multispecific antibodies (e.g. bispecific antibodies) formed from at least two intact antibodies, and antibody fragments, so long as they exhibit the desired biological activity.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al, J. Mol. Biol., 222:581-597 (1991), for example.

Antibodies specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA, 81:6851-6855 (1984)). Chimeric antibodies of interest herein include primatized antibodies comprising variable domain antigen-binding sequences derived from a non-human primate (e.g. Old World Monkey, Ape etc) and human constant region sequences.

“Antibody fragments” comprise a portion of an intact antibody, preferably comprising the antigen-binding or variable region thereof. Examples of antibody fragments include Fab, Fab′, F(ab′)₂, and Fv fragments; diabodies; linear antibodies; single-chain antibody molecules; and multispecific antibodies formed from antibody fragment(s).

An “intact” antibody is one which comprises an antigen-binding variable region as well as a light chain constant domain (C_{L}) and heavy chain constant domains, C_{H}1, C_{H}2 and C_{H}3. The constant domains may be native sequence constant domains (e.g. human native sequence constant domains) or amino acid sequence variant thereof. Preferably, the intact antibody has one or more effector functions.

“Humanized” forms of non-human (e.g., rodent) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains (Fab, Fab′, F(ab′)₂, Fabc, Fv), in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

The term “hypervariable region” when used herein refers to the regions of an antibody variable domain which are hypervariable in sequence and/or form structurally defined loops. The hypervariable region comprises amino acid residues from a “complementarity determining region” or “CDR” (i.e. residues 24-34, 50-56, and 89-97 in the light chain variable domain and 31-35, 50-65, and 95-102 in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e. residues 26-32, 50-52, and 91-96 in the light chain variable domain and 26-32, 53-55, and 96-101 in the heavy chain variable domain; Chothia and Lesk J. Mol. Biol. 196:901-917 (1987)). In both cases, the variable domain residues are numbered according to Kabat et al., supra, as discussed in more detail below. “Framework” or “FR” residues are those variable domain residues other than the residues in the hypervariable regions as herein defined.

A “parent antibody” or “wild-type” antibody is an antibody comprising an amino acid sequence which lacks one or more amino acid sequence alterations compared to an antibody variant as herein disclosed. Thus, the parent antibody generally has at least one hypervariable region which differs in amino acid sequence from the amino acid sequence of the corresponding hypervariable region of an antibody variant as herein disclosed. The parent polypeptide may comprise a native sequence (i.e. a naturally occurring) antibody (including a naturally occurring allelic variant), or an antibody with pre-existing amino acid sequence modifications (such as insertions, deletions and/or other alterations) of a naturally occurring sequence. Throughout the disclosure, “wild type,” “WT,” “wt,” and “parent” or “parental” antibody are used interchangeably.

As used herein, “antibody variant” or “variant antibody” refers to an antibody which has an amino acid sequence which differs from the amino acid sequence of a parent antibody. Preferably, the antibody variant comprises a heavy chain variable domain or a light chain variable domain having an amino acid sequence which is not found in nature. Such variants necessarily have less than 100% sequence identity or similarity with the parent antibody. In a preferred embodiment, the antibody variant will have an amino acid sequence from about 75% to less than 100% amino acid sequence identity or similarity with the amino acid sequence of either the heavy or light chain variable domain of the parent antibody, more preferably from about 80% to less than 100%, more preferably from about 85% to less than 100%, more preferably from about 90% to less than 100%, and most preferably from about 95% to less than 100%. The antibody variant is generally one which comprises one or more amino acid alterations in or adjacent to one or more hypervariable regions thereof.

An “amino acid alteration” refers to a change in the amino acid sequence of a predetermined amino acid sequence. Exemplary alterations include insertions, substitutions and deletions. An “amino acid substitution” refers to the replacement of an existing amino acid residue in a predetermined amino acid sequence; with another different amino acid residue.

A “replacement” amino acid residue refers to an amino acid residue that replaces or substitutes another amino acid residue in an amino acid sequence. The replacement residue may be a naturally occurring or non-naturally occurring amino acid residue.

An “amino acid insertion” refers to the introduction of one or more amino acid residues into a predetermined amino acid sequence. The amino acid insertion may comprise a “peptide insertion” in which case a peptide comprising two or more amino acid residues joined by peptide bond(s) is introduced into the predetermined amino acid sequence. Where the amino acid insertion involves insertion of a peptide, the inserted peptide may be generated by random mutagenesis such that it has an amino acid sequence which does not exist in nature. An amino acid alteration “adjacent a hypervariable region” refers to the introduction or substitution of one or more amino acid residues at the N-terminal and/or C-terminal end of a hypervariable region, such that at least one of the inserted or replacement amino acid residue(s) form a peptide bond with the N-terminal or C-terminal amino acid residue of the hypervariable region in question.

A “naturally occurring amino acid residue” is one encoded by the genetic code, generally selected from the group consisting of: alanine (Ala); arginine (Arg); asparagine (Asn); aspartic acid (Asp); cysteine (Cys); glutamine (Gln); glutamic acid (Glu); glycine (Gly); histidine (His); isoleucine (Ile): leucine (Leu); lysine (Lys); methionine (Met); phenylalanine (Phe); proline (Pro); serine (Ser); threonine (Thr); tryptophan (Trp); tyrosine (Tyr); and valine (Val).

A “non-naturally occurring amino acid residue” herein is an amino acid residue other than those naturally occurring amino acid residues listed above, which is able to covalently bind adjacent amino acid residues(s) in a polypeptide chain. Examples of non-naturally occurring amino acid residues include norleucine, ornithine, norvaline, homoserine and other amino acid residue analogues such as those described in Ellman et al. Meth. Enzym. 202:301-336 (1991). To generate such non-naturally occurring amino acid residues, the procedures of Noren et al. Science 244:182 (1989) and Ellman et al., supra, can be used. Briefly, these procedures involve chemically activating a suppressor tRNA with a non-naturally occurring amino acid residue followed by in vitro transcription and translation of the RNA.

Throughout this disclosure, reference is made to the numbering system from Kabat, E. A., et al., Sequences of Proteins of Immunological Interest (National Institutes of Health, Bethesda, Md. (1987) and (1991). In these compendiums, Kabat lists many amino acid sequences for antibodies for each subclass, and lists the most commonly occurring amino acid for each residue position in that subclass. Kabat uses a method for assigning a residue number to each amino acid in a listed sequence, and this method for assigning residue numbers has become standard in the field. The Kabat numbering scheme is followed in this description. For purposes of this invention, to assign residue numbers to a candidate antibody amino acid sequence which is not included in the Kabat compendium, one follows the following steps. Generally, the candidate sequence is aligned with any immunoglobulin sequence or any consensus sequence in Kabat. Alignment may be done by hand, or by computer using commonly accepted computer programs; an example of such a program is the Align 2 program. Alignment may be facilitated by using some amino acid residues which are common to most Fab sequences. For example, the light and heavy chains each typically have two cysteines which have the same residue numbers; in V_{L} domain the two cysteines are typically at residue numbers 23 and 88, and in the V_{H} domain the two cysteine residues are typically numbered 22 and 92. Framework residues generally, but not always, have approximately the same number of residues, however the CDRs will vary in size. For example, in the case of a CDR from a candidate sequence which is longer than the CDR in the sequence in Kabat to which it is aligned, typically suffixes are added to the residue number to indicate the insertion of additional residues (see, e.g. residues 100abc in FIG. 1B). For candidate sequences which, for example, align with a Kabat sequence for residues 34 and 36 but have no residue between them to align with residue 35, the number 35 is simply not assigned to a residue.

As used herein, an antibody with a “high-affinity” is an antibody having a K_{D}, or dissociation constant, in the nanomolar (nM) range or better. A K_{D} in the “nanomolar range or better” may be denoted by X nM, where X is a number less than about 10.

An “affinity matured” antibody is one with one or more alterations in one or more CDRs thereof which result an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology 10:779-783 (1992) describes affinity maturation by V_{H} and V_{L} domain shuffling. Random mutagenesis of CDR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

A “functional antigen binding site” of an antibody is one which is capable of binding a target antigen. The antigen binding affinity of the antigen binding site is not necessarily as strong as the parent antibody from which the antigen binding site is derived, but the ability to bind antigen must be measurable using any one of a variety of methods known for evaluating antibody binding to an antigen.

An antibody having a “biological characteristic” of a designated antibody is one which possesses one or more of the biological characteristics of that antibody which distinguish it from other antibodies that bind to the same antigen.

In order to screen for antibodies which bind to an epitope on an antigen bound by an antibody of interest, a routine cross-blocking assay such as that described in Antibodies, A Laboratory Manual, Cold Spring Harbor Laboratory, Ed Harlow and David Lane (1988), can be performed.

The term “filamentous phage” refers to a viral particle capable of displaying a heterogenous polypeptide on its surface, and includes, without limitation, fl, fd, Pf1, and M13. The filamentous phage may contain a selectable marker such as tetracycline (e.g., “fd-tet”). Various filamentous phage display systems are well known to those of skill in the art (see, e.g., Zacher et al. Gene 9: 127-140 (1980), Smith et al. Science 228: 1315-1317 (1985); and Parmley and Smith Gene 73: 305-318 (1988)).

The term “panning” is used to refer to the multiple rounds of screening process in identification and isolation of phages carrying compounds, such as antibodies, with high affinity and specificity to a target.

The term “short-interfering RNA (siRNA)” refers to small double-stranded RNAs that interfere with gene expression. siRNAs are an intermediate of RNA interference, the process double-stranded RNA silences homologous genes. siRNAs typically are comprised of two single-stranded RNAs of about 15-25 nucleotides in length that form a duplex, which may include single-stranded overhang(s). Processing of the double-stranded RNA by an enzymatic complex, for example by polymerases, results in the cleavage of the double-stranded RNA to produce siRNAs. The antisense strand of the siRNA is used by an RNA interference (RNAi) silencing complex to guide mRNA cleavage, thereby promoting mRNA degradation. To silence a specific gene using siRNAs, for example in a mammalian cell, the base pairing region is selected to avoid chance complementarity to an unrelated mRNA. RNAi silencing complexes have been identified in the art, such as, for example, by Fire et al., Nature 391:806-811 (1998) and McManus et al., Nat. Rev. Genet. 3(10):737-47 (2002).

The term “interfering RNA (RNAi)” is used herein to refer to a double-stranded RNA that results in catalytic degradation of specific mRNAs, and thus can be used to inhibit/lower expression of a particular gene.

The term “polymorphism” is used herein to refer to more than one forms of a gene or a portion (e.g., allelic variant) thereof. A portion of a gene of which there are at least two different forms is referred to as a “polymorphic region” of the gene. A specific genetic sequence at a polymorphic region of a gene is an “allele.” A polymorphic region can be a single nucleotide, which differs in different alleles, or can be several nucleotides long.

As used herein, the term “disorder” in general refers to any condition that would benefit from treatment with an antagonists of PirB/LILRB2, such as an anti-PirB antibody, including any condition that is expected to benefit from axon regeneration therapy, and/or an improvement of synaptic plasticity in the nervous system. Non-limiting examples of disorders to be treated herein include, without limitation, diseases and conditions benefiting from the enhancement of neurite outgrowth, promotion of neuronal growth, repair or regeneration, including neurological disorders, such as physically damaged nerves and neurodegenerative diseases. Such disorders specifically include physical damage to the central nervous system (e.g. spinal cord injury and head trauma); brain damage associated with stroke; and neurological disorders relating to neurodegeneration, such as, for example, trigeminal neuralgia, glossopharyngeal neuralgia, Bell's Palsy, myasthenia gravis, muscular dystrophy, amyotrophic lateral sclerosis (ALS), progressive muscular atrophy, progressive bulbar inherited muscular atrophy, multiple sclerosis (MS), herniated, ruptured or prolapsed invertebrate disk syndromes, cervical spondylosis, plexus disorders, thoracic outlet destruction syndromes, peripheral nerve damage caused by physical injury or disease states such as diabetes, peripheral neuropathies such as those caused by lead, dapsone, ticks, prophyria, Gullain-Barre syndrome, Alzheimer's disease, Huntington's Disease, or Parkinson's disease.

The terms “treating”, “treatment” and “therapy” as used herein refer to curative therapy, prophylactic therapy, and preventative therapy. Consecutive treatment or administration refers to treatment on at least a daily basis without interruption in treatment by one or more days. Intermittent treatment or administration, or treatment or administration in an intermittent fashion, refers to treatment that is not consecutive, but rather cyclic in nature.

The term “preventing neurodegeneration,” as used herein includes (1) the ability to inhibit or prevent neurodegeneration in patients newly diagnosed as having a neurodegenerative disease or at risk of developing a new neurodegenerative disease and (2) the ability to inhibit or prevent further neurodegeneration in patients who are already suffering from, or have symptoms of, a neurodegenerative disease.

The term “mammal” as used herein refers to any mammal classified as a mammal, including humans, higher non-human primates, rodents, domestic and farm animals, such as cows, horses, dogs and cats. In a preferred embodiment of the invention, the mammal is a human.

Administration “in combination with” one or more further therapeutic agents includes simultaneous (concurrent) and consecutive administration in any order.

An “effective amount” is an amount sufficient to effect beneficial or desired therapeutic (including preventative) results. An effective amount can be administered in one or more administrations.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. The term “progeny” refers to any and all offspring of every generation subsequent to an originally transformed cell or cell line. Mutant progeny that have the same function or biological activity as screened for in the originally transformed cell are included. Where distinct designations are intended, it will be clear from the context.

“Percent (%) amino acid sequence identity” with respect to the sequences identified herein is defined as the percentage of amino acid residues in a candidate sequence that are identical with the amino acid residues in a reference sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art can determine appropriate parameters for measuring alignment, including assigning algorithms needed to achieve maximal alignment over the full-length sequences being compared. For purposes herein, percent amino acid identity values can be obtained using the sequence comparison computer program, ALIGN-2, which was authored by Genentech, Inc. and the source code of which has been filed with user documentation in the US Copyright Office, Washington, D.C., 20559, registered under the US Copyright Registration No. TXU510087. The ALIGN-2 program is publicly available through Genentech, Inc., South San Francisco, Calif. All sequence comparison parameters are set by the ALIGN-2 program and do not vary.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to re-anneal when complementary strands are present in an environment below their melting temperature. The higher the degree of desired identity between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“High stringency conditions”, as defined herein, are identified by those that: (1) employ low ionic strength and high temperature for washing; 0.015 M sodium chloride/0.0015 M sodium citrate/0.1% sodium dodecyl sulfate at 50° C.; (2) employ during hybridization a denaturing agent; 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

A “small molecule” is defined herein to have a molecular weight below about 1000 Daltons, preferably below about 500 Daltons.

### B. Production of Anti-PirB/LILRB Antibodies

The anti-PirB/LILRB antibodies of the present invention can be produced by methods known in the art, including techniques of recombinant DNA technology.

i) Antigen Preparation

Soluble antigens or fragments thereof, optionally conjugated to other molecules, can be used as immunogens for generating antibodies. For transmembrane molecules, such as receptors, fragments of these (e.g. the extracellular domain of a receptor) can be used as the immunogen. Alternatively, cells expressing the transmembrane molecule can be used as the immunogen. Such cells can be derived from a natural source (e.g. cancer cell lines) or may be cells which have been transformed by recombinant techniques to express the transmembrane molecule. Other antigens and forms thereof useful for preparing antibodies will be apparent to those in the art.

(ii) Polyclonal Antibodies

Polyclonal antibodies are preferably raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCI₂, or R₁N═C═NR, where R and R₁ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

(iii) Monoclonal Antibodies

Monoclonal antibodies may be made using the hybridoma method first described by Kohler et al., Nature, 256:495 (1975), or may be made by recombinant DNA methods (U.S. Pat. No. 4,816,567). In the hybridoma method, a mouse or other appropriate host animal, such as a hamster or macaque monkey, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); Brodeur et al, Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

After hybridoma cells are identified that produce antibodies of the desired specificity, affinity, and/or activity, the clones may be subloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant production of antibodies will be described in more detail below.

In a further embodiment, antibodies or antibody fragments can be isolated from antibody phage libraries generated using the techniques described in McCafferty et al., Nature, 348:552-554 (1990).

Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al., Proc. Natl. Acad. Sci. USA, 81:6851 (1984)), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

(iv) Humanized and Human Antibodies

A humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987)). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the CDR residues are directly and most substantially involved in influencing antigen binding.

Alternatively, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J.sub.H) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al, Proc. Natl. Acad. Sci. USA, 90:2551 (1993); Jakobovits et al., Nature, 362:255-258 (1993); Bruggermann et al., Year in Immuno., 7:33 (1993); and Duchosal et al. Nature 355:258 (1992). Human antibodies can also be derived from phage-display libraries (Hoogenboom et al, J. Mol. Biol., 227:381 (1991); Marks et al, J. Mol. Biol., 222:581-597 (1991); Vaughan et al. Nature Biotech 14:309 (1996)). Generation of human antibodies from antibody phage display libraries is further described below.

(v) Antibody Fragments

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). In another embodiment as described in the example below, the F(ab′)₂ is formed using the leucine zipper GCN4 to promote assembly of the F(ab′)₂ molecule. According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185.

(vi) Multispecific Antibodies

Multispecific antibodies have binding specificities for at least two different epitopes, where the epitopes are usually from different antigens. While such molecules normally will only bind two different epitopes (i.e. bispecific antibodies, BsAbs), antibodies with additional specificities such as trispecific antibodies are encompassed by this expression when used herein. Examples of BsAbs include those with one arm directed against PirB/LILRB2 and another arm directed against Nogo or MAG or OMgp. A further example of BsABs include those with one arm directed against PirB/LILRB2 and another arm directed against NgR.

Methods for making bispecific antibodies are known in the art. Traditional production of full length bispecific antibodies is based on the coexpression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991). According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH₂, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in WO96/27011, the interface between a pair of antibody molecules can be engineered to maximize the percentage of heterodimers which are recovered from recombinant cell culture. The preferred interface comprises at least a part of the CH3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Fab′-SH fragments can also be directly recovered from E. coli, and can be chemically coupled to form bispecific antibodies. Shalaby et al., J. Exp. Med., 175: 217-225 (1992) describe the production of a fully humanized bispecific antibody F(ab′)₂ molecule. Each Fab′ fragment was separately secreted from E. coli and subjected to directed chemical coupling in vitro to form the bispecific antibody.

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy-chain variable domain (VH) connected to a light-chain variable domain (VL) by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fv (sFv) dimers has also been reported. See Gruber et al, J. Immunol, 152:5368 (1994).

Antibodies with more than two valencies are contemplated. For example, trispecific antibodies can be prepared. Tuft et al. J. Immunol. 147: 60 (1991).

(vii) Effector Function Engineering

It may be desirable to modify the antibody of the invention with respect to effector function, so as to enhance the effectiveness of the antibody. For example cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctonal cross-linkers as described in Wolff et al. Cancer Research 53:2560-2565 (1993). Alternatively, an antibody can be engineered which has dual Fc regions and may thereby have enhanced complement lysis and ADCC capabilities. See Stevenson et al Anti-Cancer Drug Design 3:219-230 (1989).

(viii) Antibody-Salvage Receptor Binding Epitope Fusions.

In certain embodiments of the invention, it may be desirable to use an antibody fragment, rather than an intact antibody, to increase tumor penetration, for example. In this case, it may be desirable to modify the antibody fragment in order to increase its serum half life. This may be achieved, for example, by incorporation of a salvage receptor binding epitope into the antibody fragment (e.g. by mutation of the appropriate region in the antibody fragment or by incorporating the epitope into a peptide tag that is then fused to the antibody fragment at either end or in the middle, e.g., by DNA or peptide synthesis).

The salvage receptor binding epitope preferably constitutes a region wherein any one or more amino acid residues from one or two loops of a Fc domain are transferred to an analogous position of the antibody fragment. Even more preferably, three or more residues from one or two loops of the Fc domain are transferred. Still more preferred, the epitope is taken from the CH2 domain of the Fc region (e.g., of an IgG) and transferred to the CH1, CH3, or V.sub.H region, or more than one such region, of the antibody. Alternatively, the epitope is taken from the CH2 domain of the Fc region and transferred to the CL region or VL region, or both, of the antibody fragment.

(ix) Other Covalent Modifications of Antibodies

Covalent modifications of antibodies are included within the scope of this invention. They may be made by chemical synthesis or by enzymatic or chemical cleavage of the antibody, if applicable. Other types of covalent modifications of the antibody are introduced into the molecule by reacting targeted amino acid residues of the antibody with an organic derivatizing agent that is capable of reacting with selected side chains or the N- or C-terminal residues. Examples of covalent modifications are described in U.S. Pat. No. 5,534,615, specifically incorporated herein by reference. A preferred type of covalent modification of the antibody comprises linking the antibody to one of a variety of nonproteinaceous polymers, e.g., polyethylene glycol, polypropylene glycol, or polyoxyalkylenes, in the manner set forth in U.S. Pat. No. 4,640,835; 4,496,689; 4,301,144; 4,670,417; 4,791,192 or 4,179,337.

(x) Generation of Antibodies from Synthetic Antibody Phage Libraries

In a preferred embodiment, the invention provides a method for generating and selecting novel antibodies using a unique phage display approach. The approach involves generation of synthetic antibody phage libraries based on single framework template, design of sufficient diversities within variable domains, display of polypeptides having the diversified variable domains, selection of candidate antibodies with high affinity to target the antigen, and isolation of the selected antibodies.

Details of the phage display methods can be found, for example, WO03/102157 published Dec. 11, 2003, the entire disclosure of which is expressly incorporated herein by reference.

In one aspect, the antibody libraries used in the invention can be generated by mutating the solvent accessible and/or highly diverse positions in at least one CDR of an antibody variable domain. Some or all of the CDRs can be mutated using the methods provided herein. In some embodiments, it may be preferable to generate diverse antibody libraries by mutating positions in CDRH1, CDRH2 and CDRH3 to form a single library or by mutating positions in CDRL3 and CDRH3 to form a single library or by mutating positions in CDRL3 and CDRH1, CDRH2 and CDRH3 to form a single library.

A library of antibody variable domains can be generated, for example, having mutations in the solvent accessible and/or highly diverse positions of CDRH1, CDRH2 and CDRH3. Another library can be generated having mutations in CDRL1, CDRL2 and CDRL3. These libraries can also be used in conjunction with each other to generate binders of desired affinities. For example, after one or more rounds of selection of heavy chain libraries for binding to a target antigen, a light chain library can be replaced into the population of heavy chain binders for further rounds of selection to increase the affinity of the binders.

Preferably, a library is created by substitution of original amino acids with variant amino acids in the CDRH3 region of the variable region of the heavy chain sequence. The resulting library can contain a plurality of antibody sequences, wherein the sequence diversity is primarily in the CDRH3 region of the heavy chain sequence.

In one aspect, the library is created in the context of the humanized antibody 4D5 sequence, or the sequence of the framework amino acids of the humanized antibody 4D5 sequence. Preferably, the library is created by substitution of at least residues 95-100a of the heavy chain with amino acids encoded by the DVK codon set, wherein the DVK codon set is used to encode a set of variant amino acids for every one of these positions. An example of an oligonucleotide set that is useful for creating these substitutions comprises the sequence (DVK)₇. In some embodiments, a library is created by substitution of residues 95-100a with amino acids encoded by both DVK and NNK codon sets. An example of an oligonucleotide set that is useful for creating these substitutions comprises the sequence (DVK)₆ (NNK). In another embodiment, a library is created by substitution of at least residues 95-100a with amino acids encoded by both DVK and NNK codon sets. An example of an oligonucleotide set that is useful for creating these substitutions comprises the sequence (DVK)₅ (NNK). Another example of an oligonucleotide set that is useful for creating these substitutions comprises the sequence (NNK)₆. Other examples of suitable oligonucleotide sequences can be determined by one skilled in the art according to the criteria described herein.

In another embodiment, different CDRH3 designs are utilized to isolate high affinity binders and to isolate binders for a variety of epitopes. The range of lengths of CDRH3 generated in this library is 11 to 13 amino acids, although lengths different from this can also be generated. H3 diversity can be expanded by using NNK, DVK and NVK codon sets, as well as more limited diversity at N and/or C-terminal.

Diversity can also be generated in CDRH1 and CDRH2. The designs of CDR-H1 and H2 diversities follow the strategy of targeting to mimic natural antibodies repertoire as described with modification that focus the diversity more closely matched to the natural diversity than previous design.

For diversity in CDRH3, multiple libraries can be constructed separately with different lengths of H3 and then combined to select for binders to target antigens. The multiple libraries can be pooled and sorted using solid support selection and solution sorting methods as described previously and herein below. Multiple sorting satrategies may be employed. For example, one variation involves sorting on target bound to a solid, followed by sorting for a tag that may be present on the fusion polypeptide (eg. anti-gD tag) and followed by another sort on target bound to solid. Alternatively, the libraries can be sorted first on target bound to a solid surface, the eluted binders are then sorted using solution phase binding with decreasing concentrations of target antigen. Utilizing combinations of different sorting methods provides for minimization of selection of only highly expressed sequences and provides for selection of a number of different high affinity clones.

High affinity binders for the target antigen can be isolated from the libraries. Limiting diversity in the H1/H2 region decreases degeneracy about 10⁴ to 10⁵ fold and allowing more H3 diversity provides for more high affinity binders. Utilizing libraries with different types of diversity in CDRH3 (eg. utilizing DVK or NVT) provides for isolation of binders that may bind to different epitopes of a target antigen.

Of the binders isolated from the pooled libraries as described above, it has been discovered that affinity may be further improved by providing limited diversity in the light chain. Light chain diversity is generated in this embodiment as follows in CDRL1: amino acid position 28 is encoded by RDT; amino acid position 29 is encoded by RKT; amino acid position 30 is encoded by RVW; amino acid position 31 is encoded by ANR; amino acid position 32 is encoded by THT; optionally, amino acid position 33 is encoded by CTG; in CDRL2: amino acid position 50 is encoded by KBG; amino acid position 53 is encoded by AVC; and optionally, amino acid position 55 is encoded by GMA; in CDRL3: amino acid position 91 is encoded by TMT or SRT or both; amino acid position 92 is encoded by DMC; amino acid position 93 is encoded by RVT; amino acid position 94 is encoded by NHT; and amino acid position 96 is encoded by TWT or YKG or both.

In another embodiment, a library or libraries with diversity in CDRH1, CDRH2 and CDRH3 regions is generated. In this embodiment, diversity in CDRH3 is generated using a variety of lengths of H3 regions and using primarily codon sets XYZ and NNK or NNS. Libraries can be formed using individual oligonucleotides and pooled or oligonucleotides can be pooled to form a subset of libraries. The libraries of this embodiment can be sorted against target bound to solid. Clones isolated from multiple sorts can be screened for specificity and affinity using ELISA assays. For specificity, the clones can be screened against the desired target antigens as well as other nontarget antigens. Those binders to the target antigen can then be screened for affinity in solution binding competition ELISA assay or spot competition assay. High affinity binders can be isolated from the library utilizing XYZ codon sets prepared as described above. These binders can be readily produced as antibodies or antigen binding fragments in high yield in cell culture.

In some embodiments, it may be desirable to generate libraries with a greater diversity in lengths of CDRH3 region. For example, it may be desirable to generate libraries with CDRH3 regions ranging from about 7 to 19 amino acids.

High affinity binders isolated from the libraries of these embodiments are readily produced in bacterial and eukaryotic cell culture in high yield. The vectors can be designed to readily remove sequences such as gD tags, viral coat protein component sequence, and/or to add in constant region sequences to provide for production of full length antibodies or antigen binding fragments in high yield.

A library with mutations in CDRH3 can be combined with a library containing variant versions of other CDRs, for example CDRL1, CDRL2, CDRL3, CDRH1 and/or CDRH2. Thus, for example, in one embodiment, a CDRH3 library is combined with a CDRL3 library created in the context of the humanized 4D5 antibody sequence with variant amino acids at positions 28, 29, 30, 31, and/or 32 using predetermined codon sets. In another embodiment, a library with mutations to the CDRH3 can be combined with a library comprising variant CDRH1 and/or CDRH2 heavy chain variable domains. In one embodiment, the CDRH1 library is created with the humanized antibody 4D5 sequence with variant amino acids at positions 28, 30, 31, 32 and 33. A CDRH2 library may be created with the sequence of humanized antibody 4D5 with variant amino acids at positions 50, 52, 53, 54, 56 and 58 using the predetermined codon sets.

(xi) Antibody Mutants

The novel antibodies generated from phage libraries can be further modified to generate antibody mutants with improved physical, chemical and or biological properties over the parent antibody. Where the assay used is a biological activity assay, the antibody mutant preferably has a biological activity in the assay of choice which is at least about 10 fold better, preferably at least about 20 fold better, more preferably at least about 50 fold better, and sometimes at least about 100 fold or 200 fold better, than the biological activity of the parent antibody in that assay. For example, an anti-PirB/LILRB antibody mutant preferably has a binding affinity for PirB/LILRB which is at least about 10 fold stronger, preferably at least about 20 fold stronger, more preferably at least about 50 fold stronger, and sometimes at least about 100 fold or 200 fold stronger, than the binding affinity of the parent antibody.

To generate the antibody mutant, one or more amino acid alterations (e.g. substitutions) are introduced in one or more of the hypervariable regions of the parent antibody. Alternatively, or in addition, one or more alterations (e.g. substitutions) of framework region residues may be introduced in the parent antibody where these result in an improvement in the binding affinity of the antibody mutant for the antigen from the second mammalian species. Examples of framework region residues to modify include those which non-covalently bind antigen directly (Amit et al. (1986) Science 233:747-753); interact with/effect the conformation of a CDR (Chothia et al. (1987) J. Mol. Biol. 196:901-917); and/or participate in the V_{L}-V_{H} interface (EP 239 400B1). In certain embodiments, modification of one or more of such framework region residues results in an enhancement of the binding affinity of the antibody for the antigen from the second mammalian species. For example, from about one to about five framework residues may be altered in this embodiment of the invention. Sometimes, this may be sufficient to yield an antibody mutant suitable for use in preclinical trials, even where none of the hypervariable region residues have been altered. Normally, however, the antibody mutant will comprise additional hypervariable region alteration(s).

The hypervariable region residues which are altered may be changed randomly, especially where the starting binding affinity of the parent antibody is such that such randomly produced antibody mutants can be readily screened.

One useful procedure for generating such antibody mutants is called “alanine scanning mutagenesis” (Cunningham and Wells (1989) Science 244:1081-1085). Here, one or more of the hypervariable region residue(s) are replaced by alanine or polyalanine residue(s) to affect the interaction of the amino acids with the antigen from the second mammalian species. Those hypervariable region residue(s) demonstrating functional sensitivity to the substitutions then are refined by introducing further or other mutations at or for the sites of substitution. Thus, while the site for introducing an amino acid sequence variation is predetermined, the nature of the mutation per se need not be predetermined. The ala-mutants produced this way are screened for their biological activity as described herein.

Normally one would start with a conservative substitution such as those shown below under the heading of “preferred substitutions”. If such substitutions result in a change in biological activity (e.g. binding affinity), then more substantial changes, denominated “exemplary substitutions” in the following table, or as further described below in reference to amino acid classes, are introduced and the products screened.

### Preferred Substitutions:

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; lys; arg | gln |
| Asp (D) | Glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; | leu |
| | norleucine | |
| Leu (L) | norleucine; ile; val; met; ala; | ile |
| | phe | |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; | leu |
| | norleucine | |

Even more substantial modifications in the antibodies biological properties are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr, asn, gln;

(3) acidic: asp, glu;

(4) basic: his, lys, arg;

(5) residues that influence chain orientation: gly, pro; and

(6) aromatic: trp, tyr, phe.

Non-conservative substitutions will entail exchanging a member of one of these classes for another class.

In another embodiment, the sites selected for modification are affinity matured using phage display (see above).

Nucleic acid molecules encoding amino acid sequence mutants are prepared by a variety of methods known in the art. These methods include, but are not limited to, oligonucleotide-mediated (or site-directed) mutagenesis, PCR mutagenesis, and cassette mutagenesis of an earlier prepared mutant or a non-mutant version of the parent antibody. The preferred method for making mutants is site directed mutagenesis (see, e.g., Kunkel (1985) Proc. Natl. Acad. Sci. USA 82:488).

In certain embodiments, the antibody mutant will only have a single hypervariable region residue substituted. In other embodiments, two or more of the hypervariable region residues of the parent antibody will have been substituted, e.g. from about two to about ten hypervariable region substitutions.

Ordinarily, the antibody mutant with improved biological properties will have an amino acid sequence having at least 75% amino acid sequence identity or similarity with the amino acid sequence of either the heavy or light chain variable domain of the parent antibody, more preferably at least 80%, more preferably at least 85%, more preferably at least 90%, and most preferably at least 95%. Identity or similarity with respect to this sequence is defined herein as the percentage of amino acid residues in the candidate sequence that are identical (i.e same residue) or similar (i.e. amino acid residue from the same group based on common side-chain properties, see above) with the parent antibody residues, after aligning the sequences and introducing gaps, if necessary, to achieve the maximum percent sequence identity. None of N-terminal, C-terminal, or internal extensions, deletions, or insertions into the antibody sequence outside of the variable domain shall be construed as affecting sequence identity or similarity.

Following production of the antibody mutant, the biological activity of that molecule relative to the parent antibody is determined. As noted above, this may involve determining the binding affinity and/or other biological activities of the antibody. In a preferred embodiment of the invention, a panel of antibody mutants is prepared and screened for binding affinity for the antigen or a fragment thereof. One or more of the antibody mutants selected from this initial screen are optionally subjected to one or more further biological activity assays to confirm that the antibody mutant(s) with enhanced binding affinity are indeed useful, e.g. for preclinical studies.

The antibody mutant(s) so selected may be subjected to further modifications, oftentimes depending on the intended use of the antibody. Such modifications may involve further alteration of the amino acid sequence, fusion to heterologous polypeptide(s) and/or covalent modifications such as those elaborated below. With respect to amino acid sequence alterations, exemplary modifications are elaborated above. For example, any cysteine residue not involved in maintaining the proper conformation of the antibody mutant also may be substituted, generally with serine, to improve the oxidative stability of the molecule and prevent aberrant cross linking. Conversely, cysteine bond(s) may be added to the antibody to improve its stability (particularly where the antibody is an antibody fragment such as an Fv fragment). Another type of amino acid mutant has an altered glycosylation pattern. This may be achieved by deleting one or more carbohydrate moieties found in the antibody, and/or adding one or more glycosylation sites that are not present in the antibody. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

(xii) Recombinant Production of Antibodies

For recombinant production of an antibody, the nucleic acid encoding it is isolated and inserted into a replicable vector for further cloning (amplification of the DNA) or for expression. DNA encoding the monoclonal antibody is readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence (e.g. as described in U.S. Pat. No. 5,534,615, specifically incorporated herein by reference).

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serrafia, e.g, Serratia marcescans, and Shigeila, as well as Bacilli such as B. subtilis and B. licheniformis (e.g., B. licheniformis 41P disclosed in DD 266,710 published Apr. 12, 1989), Pseudomonas such as P. aeruginosa, and Streptomyces. One preferred E. coli cloning host is E. coli 294 (ATCC 31,446), although other strains such as E. coli B, E. coli X 1776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibody are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subloned for growth in suspension culture, Graham et al, J. Gen Virol. 36:59 (1977)); baby hamster kidney cells (BHK, ATCC CCL 10); Chinese hamster ovary cells/−DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for antibody production and cultured in conventional nutrient media modified as appropriate for inducing promoters, selecting transformants, or amplifying the genes encoding the desired sequences.

The host cells used to produce the antibody of this invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. No. 4,767,704; 4,657,866; 4,927,762; 4,560,655; or 5,122,469; WO 90/03430; WO 87/00195; or U.S. Pat. No. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, as a first step, the particulate debris, either host cells or lysed cells, is removed, for example, by centrifugation or ultrafiltration. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human .gamma.1, .gamma.2, or .gamma.4 heavy chains (Lindmark et al., J. Immunol. Meth. 62:1-13 (1983)). Protein G is recommended for all mouse isotypes and for human .gamma.3 (Guss et al., EMBO J. 5:1567-1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH 3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

### C. Uses of Anti-PirB/LILRB Antibodies

The anti-PirB/LILRB antibodies of the present invention are believed to find use as agents for enhancing the survival or inducing the outgrowth of nerve cells. They are, therefore, useful in the therapy of degenerative disorders of the nervous system (“neurodegenerative diseases”), including, for example, physical damage to the central nervous system (spinal cord and brain); brain damage associated with stroke; and neurological disorders relating to neurodegeneration, such as, for example, trigeminal neuralgia, glossopharyngeal neuralgia, Bell's Palsy, myasthenia gravis, muscular dystrophy, amyotrophic lateral sclerosis (ALS), multiple sclerosis (MS), progressive muscular atrophy, progressive bulbar inherited muscular atrophy, peripheral nerve damage caused by physical injury (e.g., burns, wounds) or disease states such as diabetes, kidney dysfunction or by the toxic effects of chemotherapeutics used to treat cancer and AIDS, herniated, ruptured or prolapsed invertebrate disk syndromes, cervical spondylosis, plexus disorders, thoracic outlet destruction syndromes, peripheral neuropathies such as those caused by lead, dapsone, ticks, prophyria, Gullain-Barre syndrome, Alzheimer's disease, Huntington's Disease, or Parkinson's disease.

The anti-PirB/LILRB antibodies herein are also useful as components of culture media for use in culturing nerve cells in vitro.

Finally, preparations comprising the anti-PirB/LILRB antibodies herein are useful as standards in competitive binding assays when labeled with radioiodine, enzymes, fluorophores, spin labels, and the like.

Therapeutic formulations of the anti-PirB/LILRB antibodies herein are prepared for storage by mixing the compound identified (such as an antibody) having the desired degree of purity with optional physiologically acceptable carriers, excipients or stabilizers (Remington's Pharmaceutical Sciences, supra), in the form of lyophilized cake or aqueous solutions. Acceptable carriers, excipients or stabilizers are nontoxic to recipients at the dosages and concentrations employed, and include buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine or lysine; monosaccharides, disaccharides and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or PEG.

The anti-PirB/LILRB antibodies to be used for in vivo administration must be sterile. This is readily accomplished by filtration through sterile filtration membranes, prior to or following lyophilization and reconstitution.

Therapeutic compositions may be placed into a container having a sterile access port, for example, an intravenous solution bag or vial having a stopper pierceable by a hypodermic injection needle.

The anti-PirB/LILRB antibodies of the present invention may be optionally combined with or administered in combination with neurotrophic factors including NGF, NT-3, and/or BDNF and used with other conventional therapies for degenerative nervous disorders. In addition, the anti-PirB/LILRB antibodies of the present invention can be advantageously administered in combination with NgR inhibitors, such as antibodies, small molecules or peptides, blocking the binding of Nogo-66, MAG and/or OMgp to NgR.

The route of administration is in accord with known methods, e.g. injection or infusion by intravenous, intraperitoneal, intracerebral, intramuscular, intraocular, intraarterial or intralesional routes, topical administration, or by sustained release systems as noted below.

For intracerebral use, the compounds may be administered continuously by infusion into the fluid reservoirs of the CNS, although bolus injection is acceptable. The compounds are preferably administered into the ventricles of the brain or otherwise introduced into the CNS or spinal fluid. Administration may be performed by an indwelling catheter using a continuous administration means such as a pump, or it can be administered by implantation, e.g., intracerebral implantation, of a sustained-release vehicle. More specifically, the compounds can be injected through chronically implanted cannulas or chronically infused with the help of osmotic minipumps. Subcutaneous pumps are available that deliver proteins through a small tubing to the cerebral ventricles. Highly sophisticated pumps can be refilled through the skin and their delivery rate can be set without surgical intervention. Examples of suitable administration protocols and delivery systems involving a subcutaneous pump device or continuous intracerebroventricular infusion through a totally implanted drug delivery system are those used for the administration of dopamine, dopamine agonists, and cholinergic agonists to Alzheimer patients and animal models for Parkinson's disease described by Harbaugh, J. Neural Transm. Suppl., 24:271 (1987); and DeYebenes, et al., Mov. Disord. 2:143 (1987).

Suitable examples of sustained release preparations include semipermeable polymer matrices in the form of shaped articles, e.g. films, or microcapsules. Sustained release matrices include polyesters, hydrogels, polylactides (U.S. Pat. No. 3,773,919, EP 58,481), copolymers of L-glutamic acid and gamma ethyl-L-glutamate (Sidman, et al., 1983, Biopolymers 22:547), poly (2-hydroxyethyl-methacrylate) (Langer, et al., 1981, J. Biomed. Mater. Res. 15:167; Langer, 1982, Chem. Tech. 12:98), ethylene vinyl acetate (Langer, et al., Id.) or poly-D-(−)-3-hydroxybutyric acid (EP 133,988A) Sustained release compositions also include liposomally entrapped compounds, which can be prepared by methods known per se. (Epstein, et al., Proc. Natl. Acad. Sci. 82:3688 (1985); Hwang, et al., Proc. Natl. Acad. Sci. USA 77:4030 (1980); U.S. Pat. Nos. 4,485,045 and 4,544,545; and EP 102,324A). Ordinarily the liposomes are of the small (about 200-800 Angstroms) unilamelar type in which the lipid content is greater than about 30 mol. % cholesterol, the selected proportion being adjusted for the optimal therapy.

An effective amount of an active compound to be employed therapeutically will depend, for example, upon the therapeutic objectives, the route of administration, and the condition of the patient. Accordingly, it will be necessary for the therapist to titer the dosage and modify the route of administration as required to obtain the optimal therapeutic effect. A typical daily dosage might range from about 1 μg/kg to up to 100 mg/kg or more, depending on the factors mentioned above. Typically, the clinician will administer an active compound until a dosage is reached that repairs, maintains, and, optimally, reestablishes neuron function. The progress of this therapy is easily monitored by conventional assays.

Further details of the invention are illustrated by the following non-limiting examples.

### Example 1

### Expression Cloning LILRB2

To identify novel receptors for inhibitory myelin proteins, an expression cloning approach was taken. As bait, constructs were generated that fused Alkaline Phosphatase (AP) to the N- and/or C-terminus of the following characterized myelin inhibitors (human cDNA used): Nogo66, two additional inhibitory domains of NogoA (NiR<delta>D2 and NiG<delta>20) (Oertle T, J Neurosci. 2003, 23(13): 5393-406), MAG, and OMgp. These constructs were transfected into 293 cells to produce conditioned medium (in DMEM/2% FBS) containing the bait proteins. The cDNA library used in the screen was comprised of full-length human cDNA clones in expression-ready vectors generated by Origene. These cDNAs were compiled, arrayed, and pooled. Pools of approximately 100 cDNA's were transiently transfected into COS7 cells.

In particular, on Day 1, COS7 cells were plated at a density of 85, 000 cells per well in 12-well plates. On Day 2, 1 mg of pooled cDNA's were transfected per well using the lipid-based transfection reagent FuGENE 6 (Roche). On Day 4, screening was performed. Briefly, culture medium was removed from cells and replaced with 0.5 ml of 293 cell-conditioned medium containing AP-fusion bait proteins (20-50 nM). Cells were incubated at room temperature for 90 minutes. The cells were then washed 3 times with phosphate-buffered saline (PBS), fixed for 7 minutes with 4% paraformaldehyde, washed 3 times in HEPES-buffered saline (HBS), and heat inactivated at 65° C. for 90 minutes to destroy endogenous AP activity. The cells were washed once in AP Buffer (100 mM NaCl, 5 mM MgCl₂, 100 mM Tris pH 9.5), and incubated in chromogenic substrate (Western Blue, Promega), and analyzed for presence of reaction product one hour after incubation, and again after overnight incubation. Positive cells were identified by the presence of dark blue precipitate over the surface of the membrane. Positive pools were further broken down to identify individual positive clones by subsequent rounds of screening.

From the screening, the following positive hits were identified:

MAG-AP bait yielded 4 positive hits. One was the previously characterized Nogo Receptor (Fournier et al., Nature 409, 342-346 (2001). Two of these hits were glycolytic processing enzymes, and deemed unlikely to be of relevance. The fourth was annotated as “Homo sapiens hypothetical protein from clone 643 (LOC57228), mRNA”. Closer analysis of the cDNA revealed an alternative ORF that was homologous to the previously described protein SMAG.

AP-Nogo66 bait yielded 2 positive hits. One was the previously characterized Nogo Receptor. The other was “Homo sapiens leukocyte immunoglobulin-like receptor, subfamily B (with TM and ITIM domains), member 2 (LILRB2), mRNA” (SEQ ID NO: 2). This gene is also known by multiple alternative nomenclatures, including MIG10, ILT4, and LIR2 (Kubagawa et al., Proc. Natl. Acad. Sci. USA 94:5261-6 (1997); Colonna et al., J. Exp. Med. 186:1809-18 (1997)).

### Example 2

### Preparation and Testing of PirB Function-Blocking Antibodies

PirB Function-Blocking Antibodies

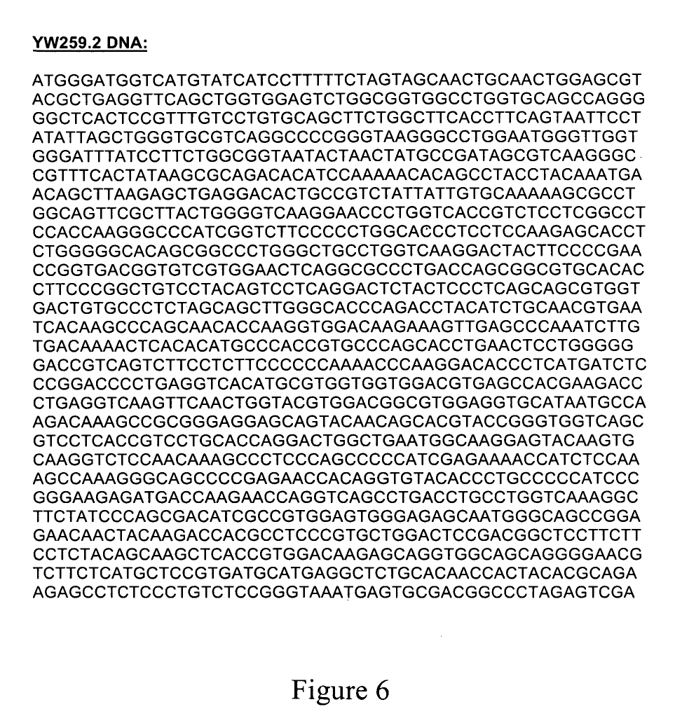

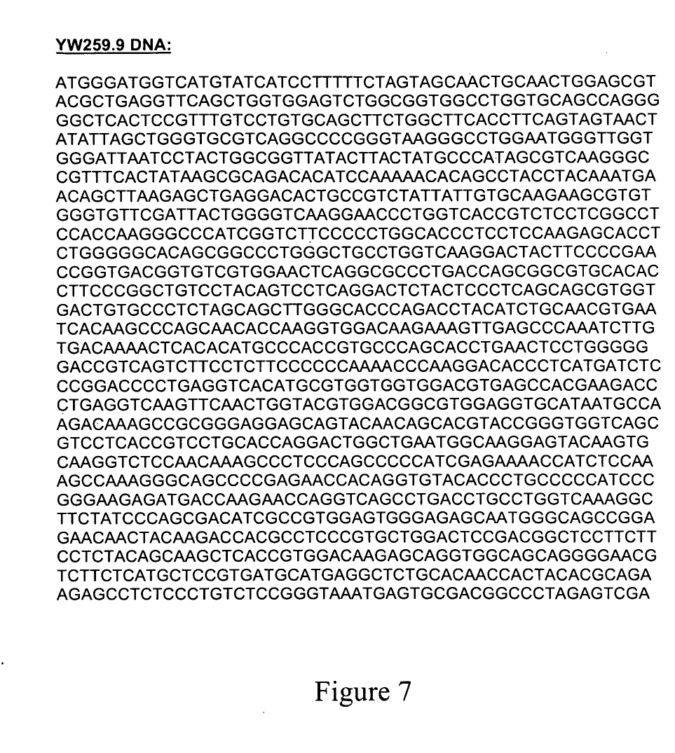

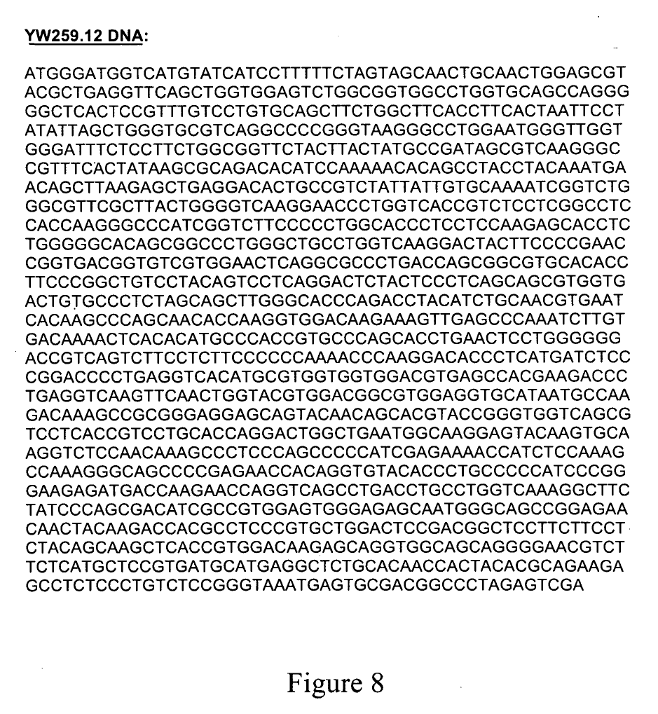

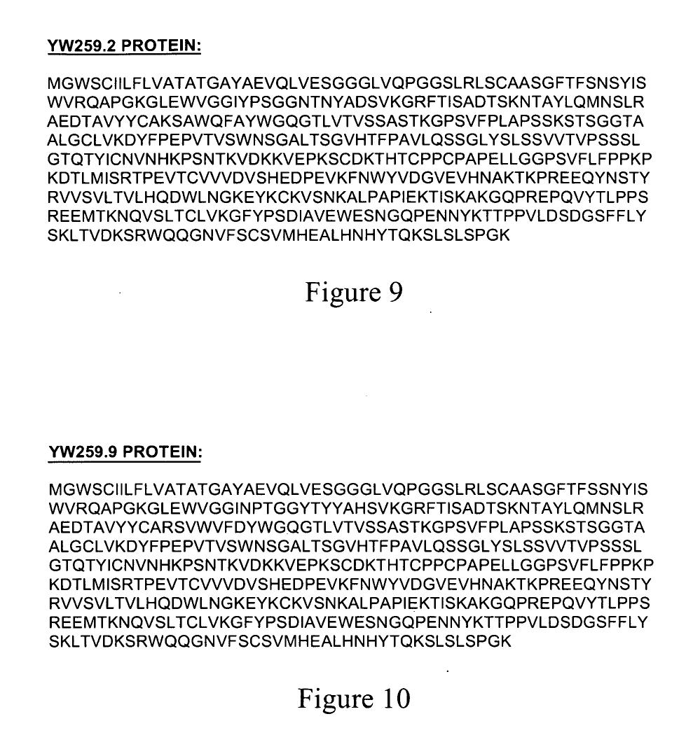

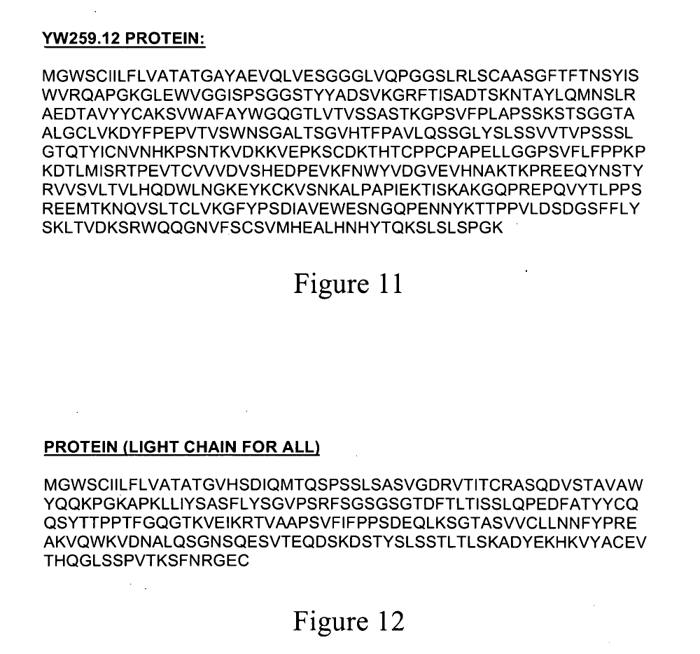

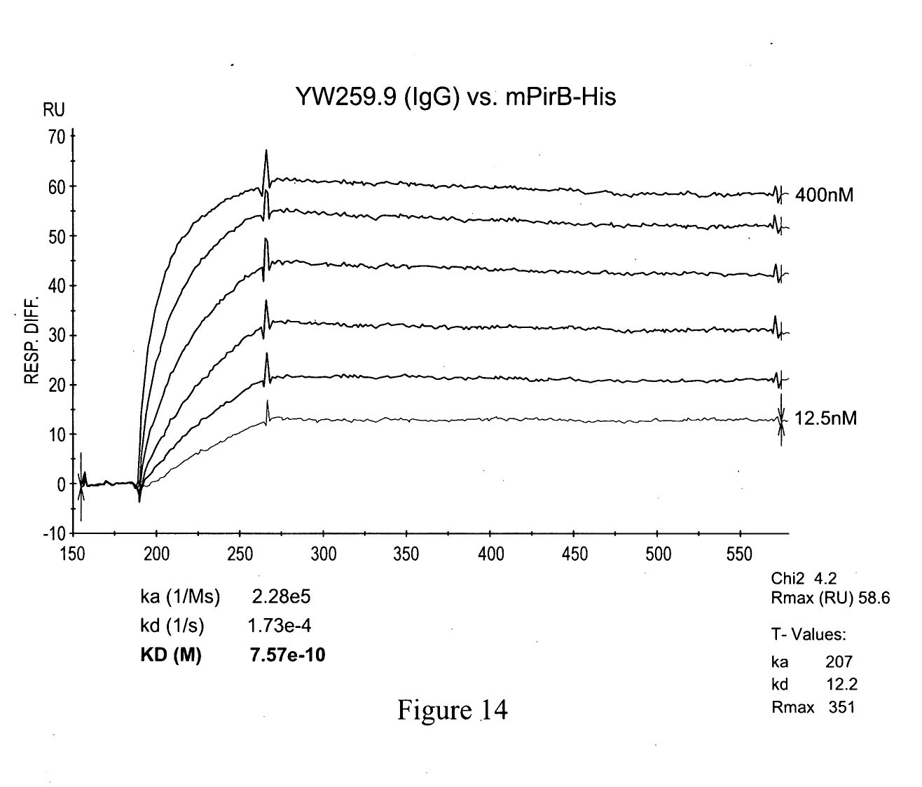

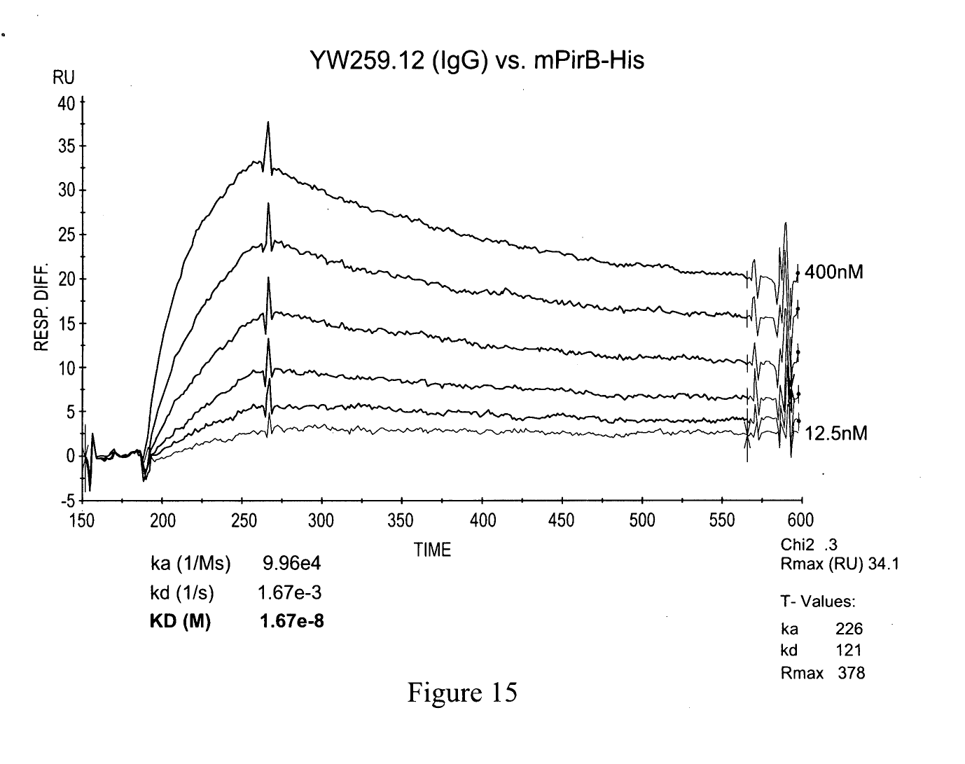

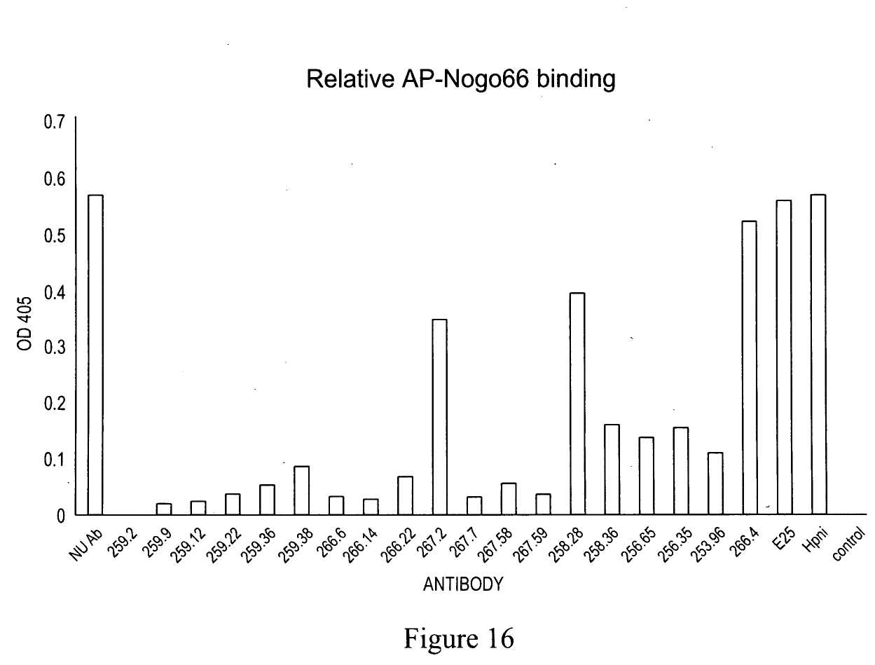

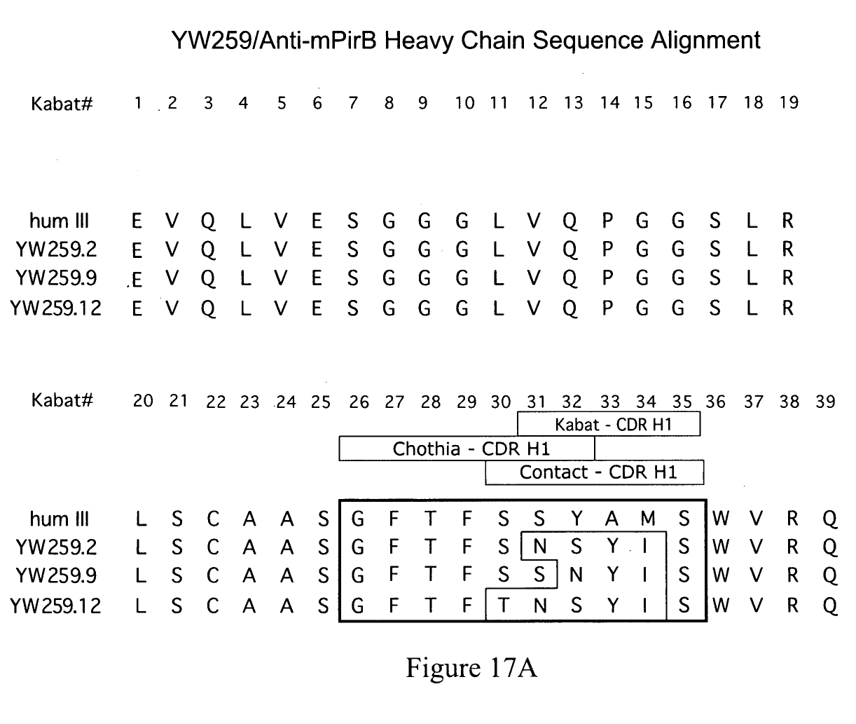

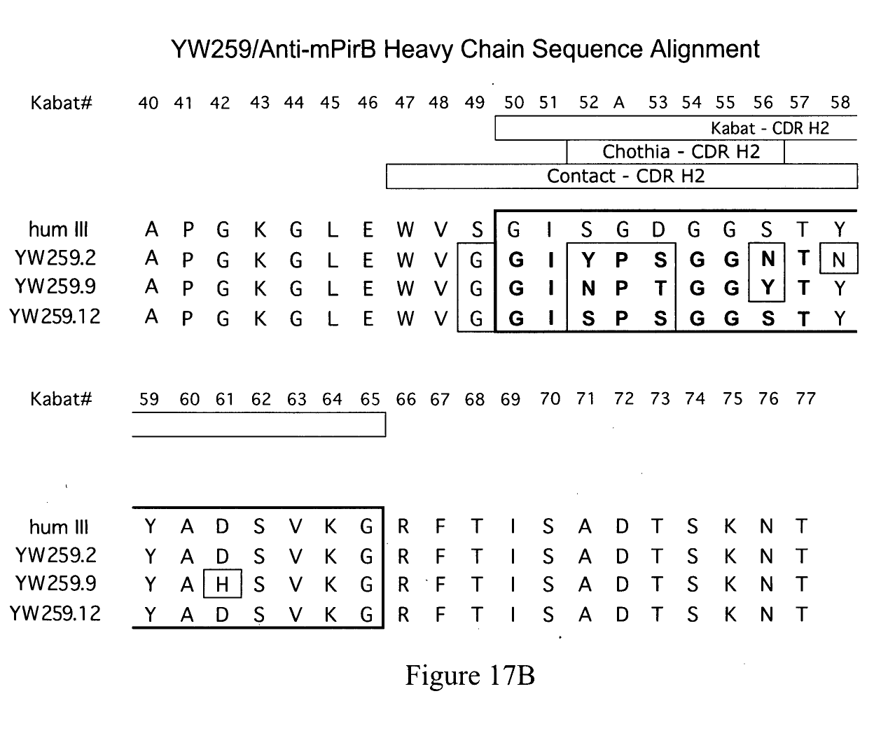

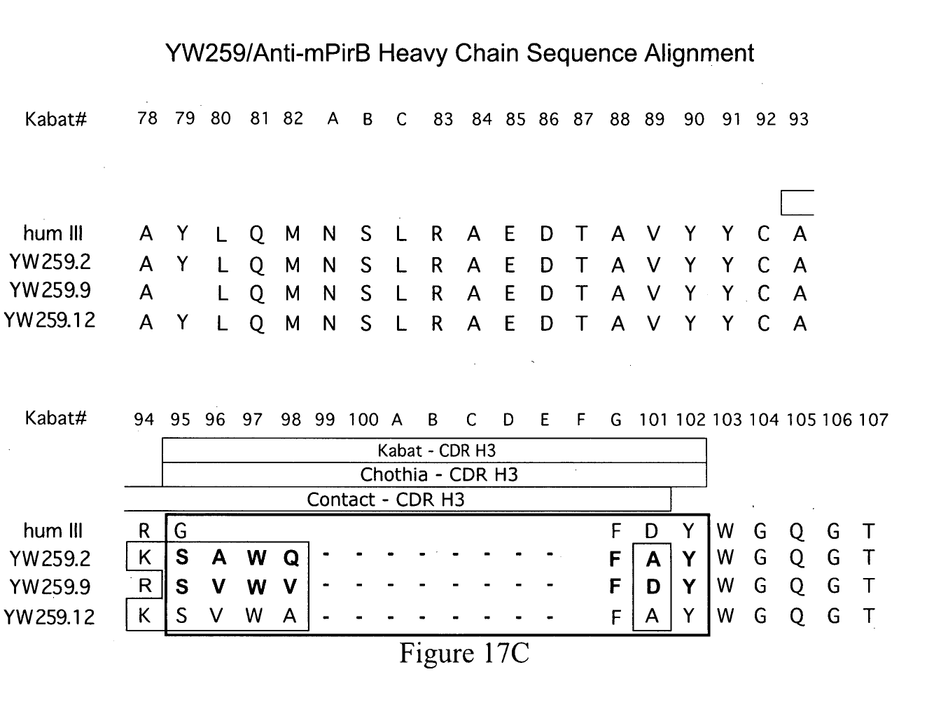

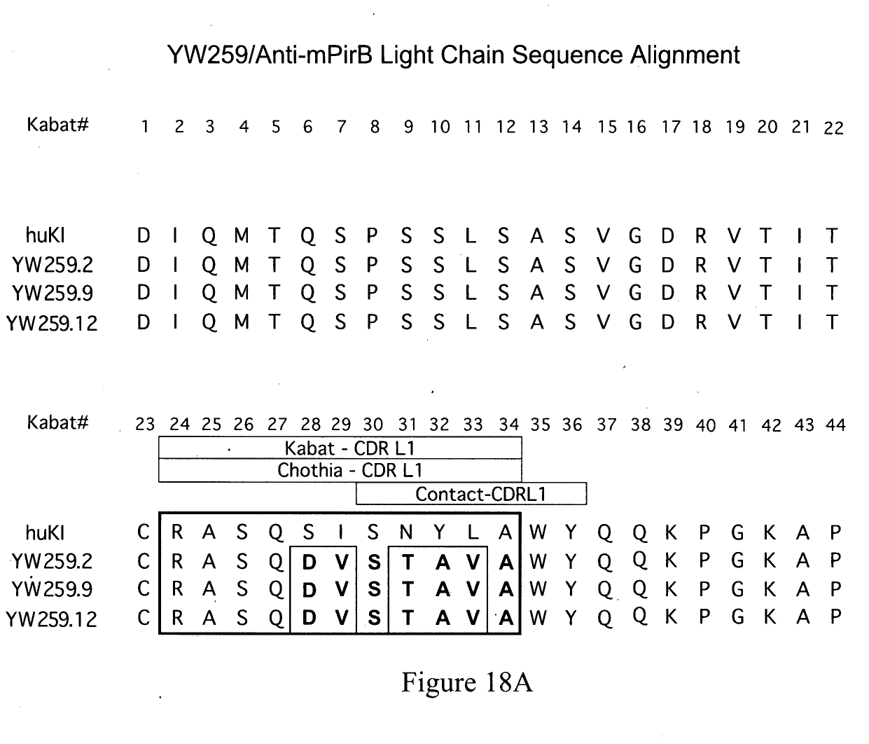

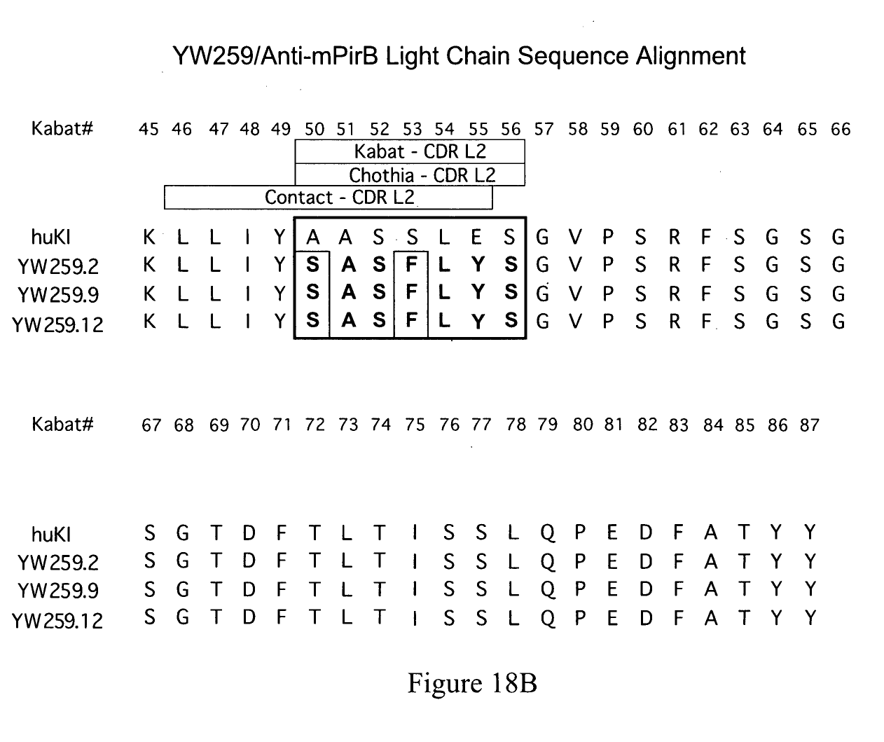

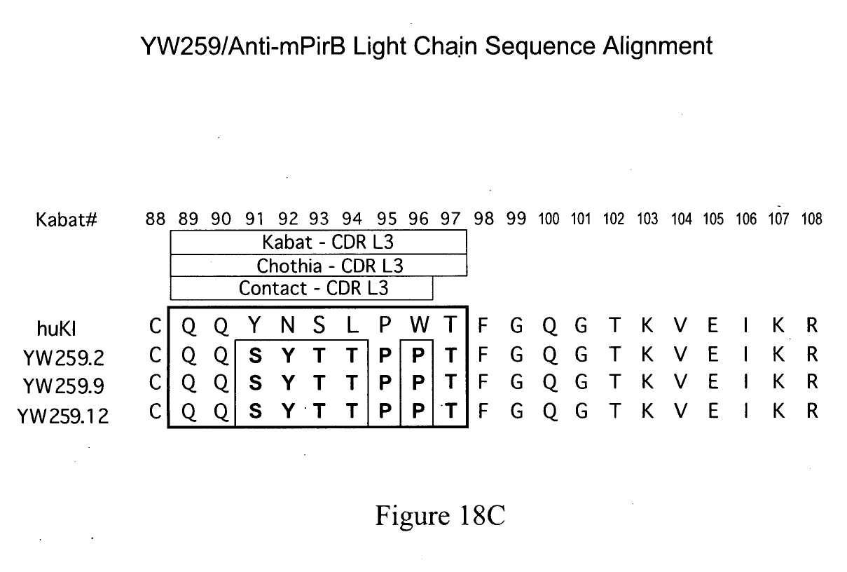

Antibodies against PirB were generated by panning a synthetic phage antibody library against the PirB extracellular domain (W. C. Liang et al., J Mol Biol 366, 815 (2007)). Antibody clones (10 μg/ml) were then tested in vitro for their ability to block binding of AP-Nogo66 (50 nM) to PirB-expressing COS7 cells. The nucleotide and amino acid sequences of the heavy and light chain sequences of various YW259 anti-mouse PirB (anti-mPirB) antibodies are shown in FIGS. 6-16, and 17 and 18. FIGS. 17 and 18 also show the hypervariable region sequences within the heavy and light chains of YW259.2, YW250.9 and YW259.12, respectively.

Neurite Outgrowth Assay

96-well plates pre-coated with poly-D-lysine (Biocoat, BD) were coated with myelin (0.75 μg/ml) overnight or with AP-Nogo66 or MAG-Fc (150-300 ng/spot) for two hours, and then treated with laminin (10 μg/ml in F-12) for 2 hours (CGN cultures) or 4 hours (DRG cultures). Mouse P7 cerebellar neurons were cultured as previously described (B. Zheng et al., Proc Natl Acad Sci USA 102, 1205 (2005)) and plated at ˜2×10⁴ cells per well. Mouse P10 DRG neurons were cultured as previously described (Zheng et al., 2005, supra) and plated at ˜5×10³ cells per well. Cultures were grown for 22 hours at 37° C. with 5% CO₂, and then fixed with 4% paraformaldehyde/10% sucrose and stained with anti-βIII-tubulin (TuJ1, Covance). For each experiment, all conditions were performed in six replicate wells, from which maximum neurite lengths were measured and averages were determined between the six wells. Each experiment was performed at least three times with similar results. p-values were determined using Student's t test.

Growth Cone Collapse Assay

DRG explants were isolated by dissecting out DRG from 3-week-old mice and slicing them into thirds. Each DRG explant was then cultured in an individual PDL (100 μg/ml)- and laminin (10 μg/ml)-coated well from an eight-well plate. At 72-hours-post-plating, explants were incubated with AP-Nogo66 (100 nM) or myelin (3 μg/ml) for 30 minutes to stimulate collapse. Cultures were fixed with 4% paraformaldehyde/10% sucrose, and growth cones were then visualized by rhodamine-phalloidin (Molecular Probes) staining and scored for collapse. Average growth cone collapse was determined by averaging at least 3 replicate wells.

Results

To address whether PirB is a functional receptor for Nogo66, we focused on juvenile (P7) cerebellar granule neurons (CGN), for which neurite outgrowth is inhibited when grown on AP-Nogo66 (K. C. Wang et al., Nature 420, 74 (2002)). Adult CGN have been shown to express PirB (J. Syken et al., Science 313,1795 (2006)), and we found that is also the case for juvenile CGN as assessed by RT-PCR, immunohistochemistry and in situ hybridization (data not shown).

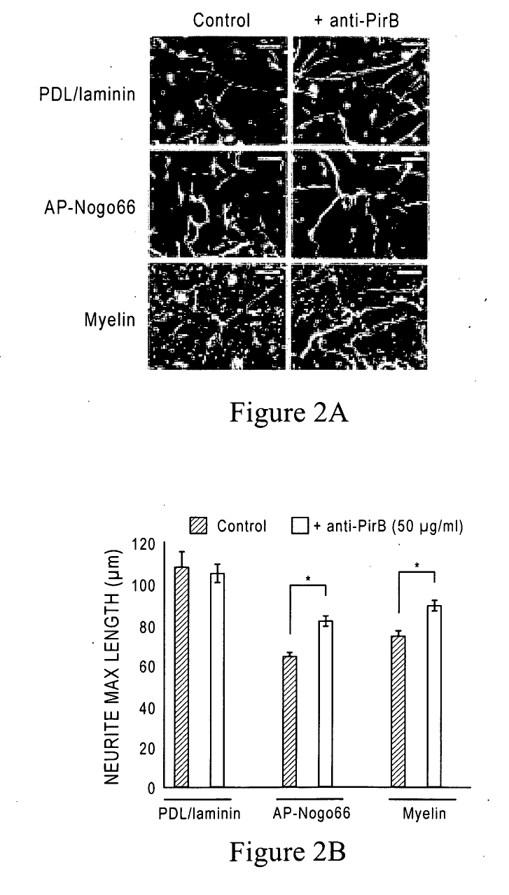

First, the ability of a soluble ectodomain of PirB (PirB-His) to interfere with AP-Nogo66 inhibition was tested in vitro. As shown in FIG. 2A, AP-Nogo66 inhibits neurite outgrowth of P7 CGN to approximately 66% of untreated control levels. Inclusion of PirB-His in this assay reversed AP-Nogo66 inhibition, with neurite outgrowth returning essentially to control levels. These results are similar to those reported using the ectodomain of NgR to block inhibition by Nogo66 (B Zgeng et al., Proc. Natl. Acad. Sci. USA 102, 1205 (2005); A. E. Fournier, et al., J. Neurosci. 22, 8876 (200); Z. L. He et al., Neuron 38, 177 (2003)), and indicate that PirB can bind the functionally inhibitory domain of Nogo66, but do not address whether endogenous PirB in CGN mediates inhibition by AP-Nogo66.

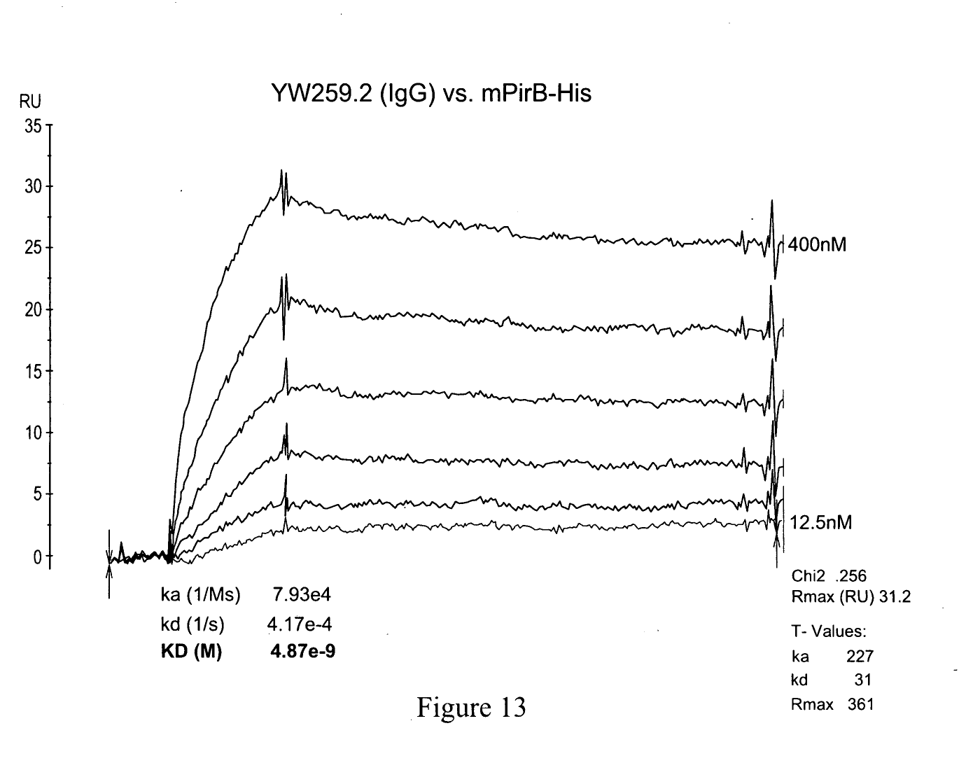

Therefore, antibodies were generated to PirB (anti-PirB) capable of interfering with the PirB-Nogo66 interaction. Using a phage display platform (W. C. Liang et al., J. Mol. Biol. 366, 815 (2007)) directed against the extracellular domain of PirB, multiple clones were screened for their ability to block binding of AP-Nogo66 to PirB. Clone YW259.2 (hereafter referred to as aPB1), which interfered best with AP-Nogo66-PirB binding, had a Kd of 5 nM for PirB (see, FIGS. 13-16).

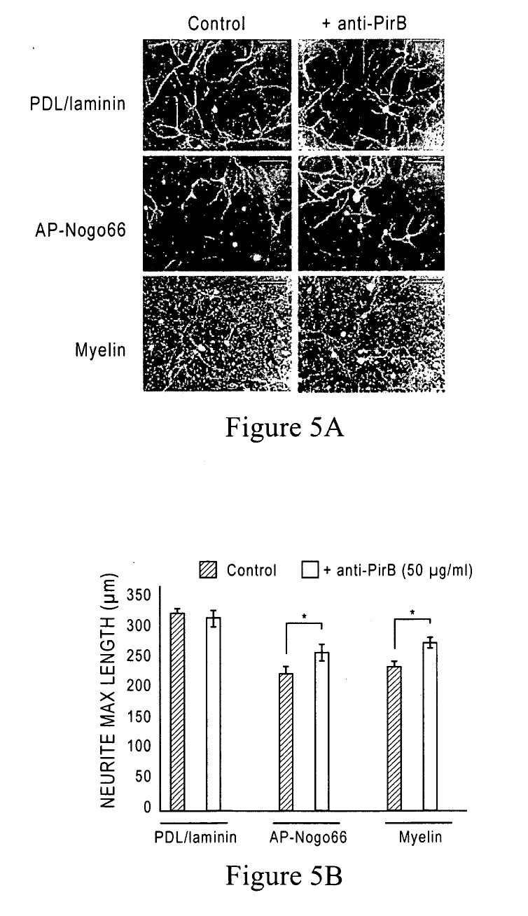

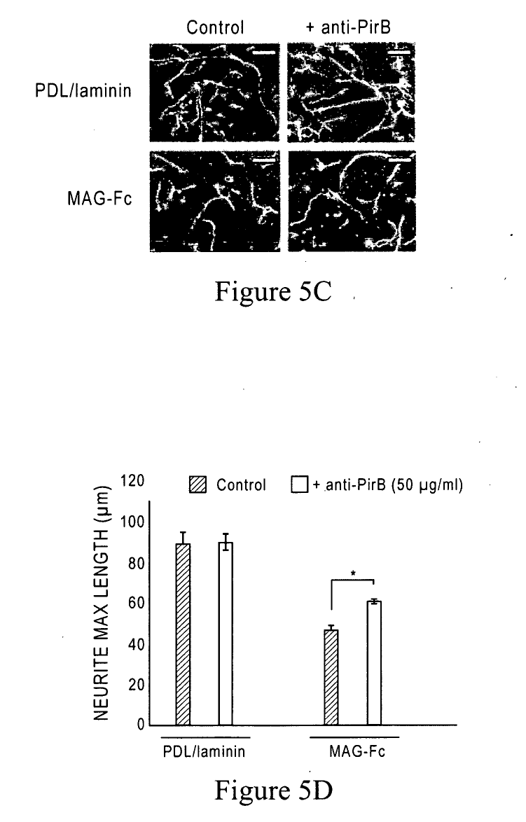

aPB1 had no effect on the baseline axon growth of CGNs. However, aPB1 significantly reduced inhibition by AP-Nogo66 or myelin in cultured CGN (FIG. 2B), rescuing neurite outgrowth to 59% from 41% on AP-Nogo66, and 62% from 47% on myelin. Similar results were seen using MAG as an inhibitory substrate, or using a different cell type (dorsal root ganglion (DRG) neurons) (FIG. 5). These results argue that PirB is a functional receptor mediating long-term inhibition of neurite outgrowth.

To confirm this result, it was tested whether genetic removal of cell surface PirB also reversed inhibition by AP-Nogo66 or myelin, by culturing neurons from PirB™ mice, in which four exons encoding the transmembrane domain and part of the PirB intracellular domain have been removed (J. Syken et al., Science 313,1795 (2006))). CGN were cultured from PirB™ mice or wild-type (WT) littermates on control substrate, AP-Nogo66 or myelin. On control substrate (PDL/laminin), PirB™ neurons behaved similarly to WT neurons (FIG. 2C). However, neurite outgrowth from PirB™ neurons was markedly less inhibited than from WT neurons on either AP-Nogo66 or myelin. On AP-Nogo66, outgrowth from WT neurons was inhibited to 50% of control levels, whereas PirB™ neurons were inhibited to only 66%. Similarly, on myelin, WT neurons were inhibited to 52% of control levels, whereas PirB™ neurons were inhibited to only 70%. Again, we saw similar partial disinhibition of PirB™ DRG neurons on both myelin and AP-Nogo66 (FIG. 5). These findings indicate that PirB is indeed a functional receptor for AP-Nogo66 and myelin-mediated inhibition of neurite growth. However, loss of PirB activity does not fully rescue outgrowth.

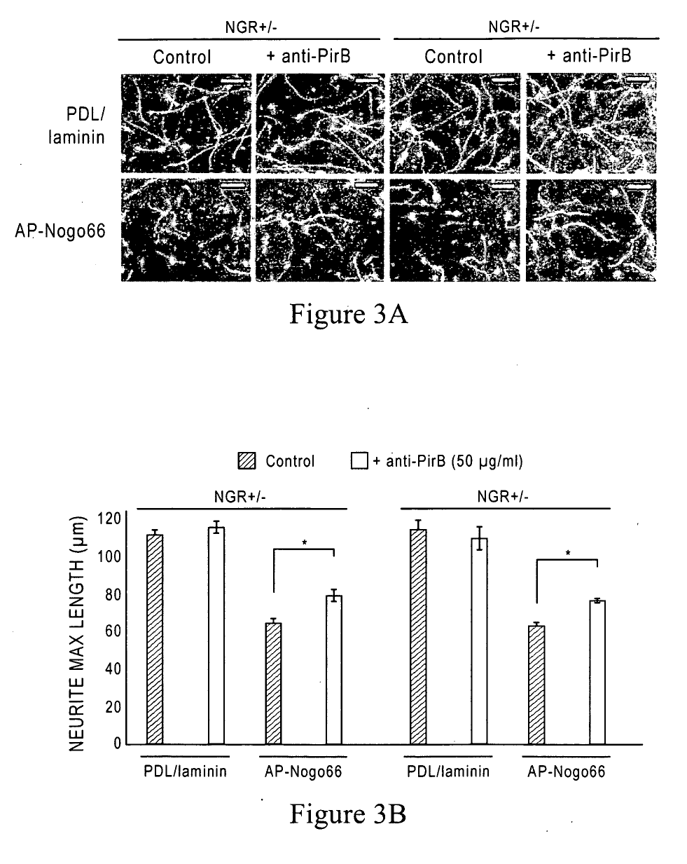

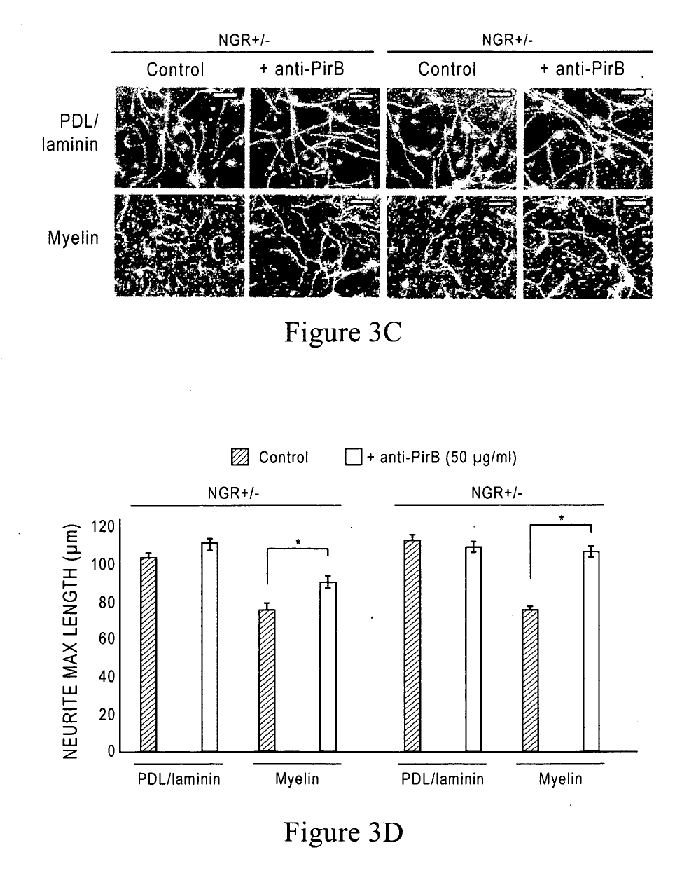

Since NgR has previously been described as a receptor for myelin inhibitors, it is possible that PirB and NgR function together to mediate inhibition of neurite outgrowth. To address this, both PirB and NgR function were blocked together in CGN's by culturing neurons from NgR-null mice in the presence of anti-PirB. As we have reported previously (B. Cheng et al., PNAS 2005, supra), NgR−/− CGN neurite outgrowth is inhibited by AP-Nogo66 or myelin to the same extent as that in WT neurons (50% and 49%; FIG. 3). aPB1 antibody treatment of NgR+/− neurons partially reversed inhibition by either AP-Nogo66 or myelin, as seen above for aPB1 treatment of WT neurons. Similarly, aPB1 treatment of NgR−/− neurons partially reversed inhibition by AP-Nogo66, but did not provide any further rescue than that seen with aPB1 treatment of NgR+/− neurons or WT neurons. In contrast, aPB1 treatment of NgR−/− neurons restored neurite outgrowth on myelin to nearly control levels. Thus, it appears that PirB, but not NgR, is required for substrate inhibition by APNogo66 in CGN, but only in part. Moreover both PirB and NgR together contribute to the substrate inhibition imparted by myelin.

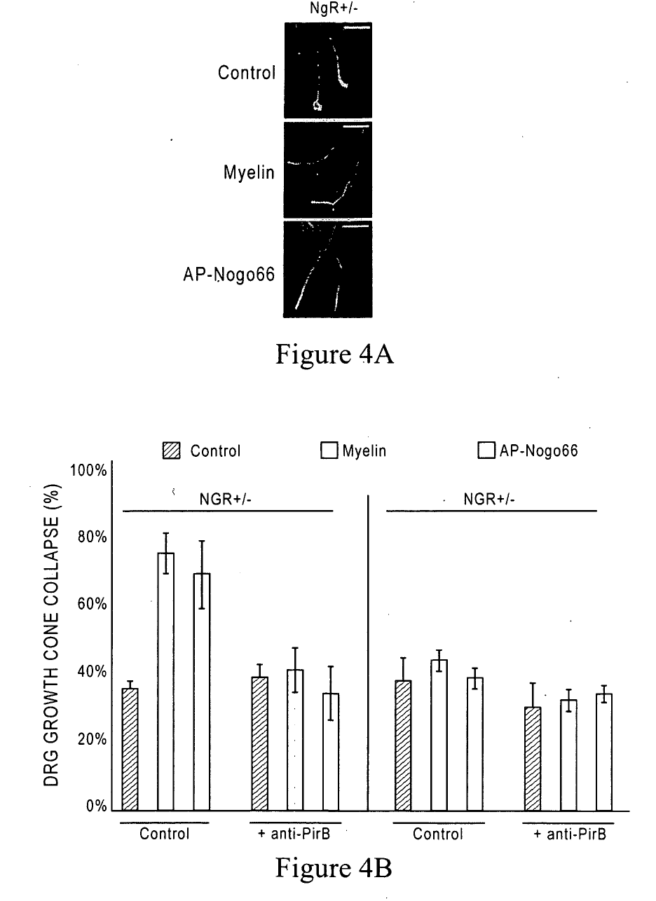

Since NgR is thought to be required for growth cone collapse in response to various myelin inhibitors (J. E. Kim et al., Neuron 44, 439 (2004), O. Chivatakarn et al., J. Neurosci. 27, 7117 (2007)), it is possible that PirB is also involved in this more acute response. Sensory neurons from the dorsal root ganglia (DRG) of 3-week-old mice, confirmed to express PirB, were used for this experiment. It has been found that growth cones in this culture system have a high baseline level of collapse (˜30%), which is further increased by incubation with APNogo66 or myelin (FIG. 4). This collapse was largely abolished in NgR−/− neurons. In addition, blocking PirB function with aPB1 was also sufficient to reverse growth cone collapse by these inhibitors. Inhibiting both PirB and NgR pathways together (using aPB1 treatment on neurons from NgR−/− mice) also fully reversed growth cone collapse, but this result was not informative since either treatment alone gave full rescue in this assay.

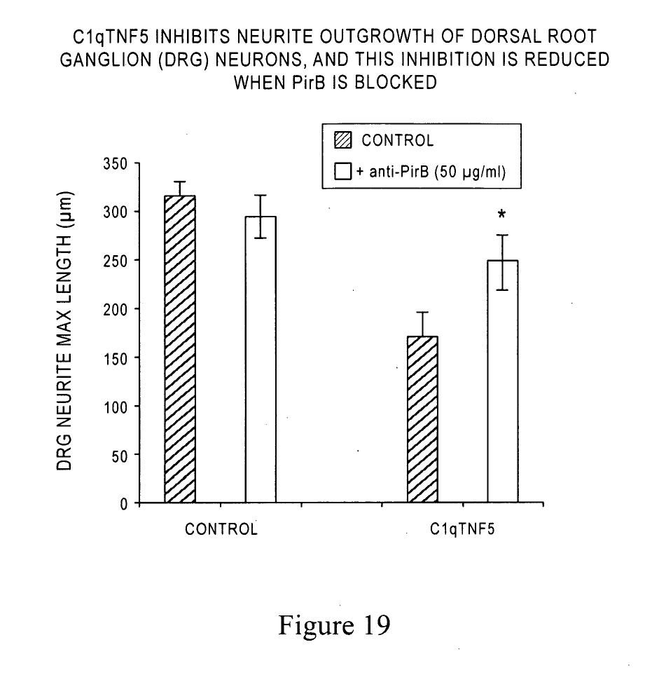

In another experiment, C1QTNF5 inhibited neurite outgrowth of cereberral granule neurone (CGN), and this inhibition was reversed by PirB function-blocking antibody YW259.2. The results are shown in FIG. 19.

Together, these results support a novel role for PirB as a necessary receptor for neurite inhibition by myelin extracts, and more specifically by the myelin-associated inhibitors Nogo66 and MAG. Indeed, PirB appears to be a more significant mediator of substrate inhibition than NgR, since removal of PirB function alone (either genetically or using antibodies) partially disinhibits growth on both myelin extracts and myelin inhibitors, whereas genetic removal of NgR alone does not disinhibit on any of these substrates. However, NgR appears to play an adjunct role in mediating inhibition by myelin extracts (but not Nogo66), since genetic removal of NgR can augment the disinhibition caused by anti-PirB antibodies on myelin (but not on Nogo66). Our findings may help to explain the surprising lack of enhanced CST regeneration in NgR knockout mice (J. E. Kim et al., supra, B. Zheng et al., Proc. Natl. Acad. Sci. USA 102, 1205 (2005)), despite the reported regeneration or sprouting seen in rodents infused with the NgR ectodomain (S. Li et al., J. Neurosci. 24, 10511 (2004)). Thus, it might be necessary to remove both PirB and NgR to achieve significant regeneration in vivo. In addition, since on Nogo66 substrate the genetic removal of NgR does not further augment the partial disinhibitory effect of PirB removal, it is likely that there are additional binding receptor(s) for Nogo66.

Although PirB appears to be a more significant receptor for substrate inhibition than NgR, inactivation of either PirB or NgR alone is sufficient to block the acute growth cone collapse caused by addition of myelin inhibitors. This observation suggests that collapse is a more demanding process, requiring both PirB and NgR activities, acting either in parallel or together. In this context, it is of interest that PirB and NgR receptors have recently been shown to play similar roles in limiting plasticity of synaptic connections in the visual cortex: in mice lacking either receptor, eye closure during a critical developmental period results in excessive strengthening of connections via the open eye (J. Syken et al., 2006, supra, A. W. McGee et al., Science 309, 2222 (2005), supra). The mechanisms responsible for the effect of both receptors in mediating growth cone collapse could also underlie the commonality of their role in ocular dominance plasticity.

The inability of adult axons to regenerate following injury is a major obstacle to regaining function after traumatic insults to the CNS. It has been speculated that regeneration potential declines as the capacity for synaptic plasticity becomes limited with age, in an effort to restrict the development of excess or exuberant synaptic connections. This speculation gains support from the finding that PirB, previously implicated in limiting synaptic plasticity both during development and in adulthood (J. Syken et al., 2006, supra), is also a mediator of axonal inhibition by myelin, providing a parallel with the finding that NgR, initially implicated in axonal inhibition, similarly regulates synaptic plasticity (S. Li et al., J. Neurosci. 24, 10511 (2004)).

Our findings also broaden the repertoire of potential PirB ligands beyond the scope of Class I MHC molecules, to include neuronal regrowth inhibitors. Conversely, since genetic deletion of the known myelin inhibitor Nogo or MAG results in only a modest decrease in inhibition by myelin—implying that other inhibitors are present—our findings raise the possibility that MHCI molecules, which are normally expressed at low levels by oligodendrocytes, may be upregulated following injury and contribute to outgrowth inhibition in concert with Nogo and MAG in central myelin.

The mechanism by which PirB signals to inhibit axon growth in response to myelin inhibitors is not clear. However, PirB has been shown to antagonize the function of integrin receptors (S. Pereira et al., J. Immunol. 173:5757 (2004)), and to recruit both SHP-1 and SHP-2 phosphatases); either or both of these events could attenuate normal neurite outgrowth. Blockade of PirB activity, using the anti-PirB antibodies herein or by other means, provides an important new target for therapeutic interventions to stimulate axonal regeneration.

All references cited throughout the disclosure are hereby expressly incorporated by reference in their entirety

While the present invention has been described with reference to what are considered to be the specific embodiments, it is to be understood that the invention is not limited to such embodiments. To the contrary, the invention is intended to cover various modifications and equivalents included within the spirit and scope of the appended claims.

What is claimed is:

1. An isolated anti-PirB/LILRB antibody that binds to a same epitope on human PirB (LILRB) as an antibody selected from the group consisting of YW259.2, YW259.9 and YW259.12.

2. An isolated anti-PirB/LILRB antibody that competes for binding to human PirB (LILRB) with an antibody selected from the group consisting of YW259.2, YW259.9 and YW259.12.

3. An isolated anti-PirB/LILRB antibody that comprises one, two, or three hypervariable region sequences from a heavy chain selected from the group consisting of: YW259.2 heavy chain (SEQ ID NO: 4 or 11), YW259.9 heavy chain (SEQ ID NO: 5 or 12), and YW259.12 heavy chain (SEQ ID NO: 6 or 13).

4. The antibody of claim 3 wherein the antibody comprises all hypervariable region sequences of the YW259.2 antibody heavy chain (SEQ ID NO: 4 or 11).

5. The antibody of claim 3 wherein the antibody comprises all hypervariable region sequences of the YW259.9 antibody heavy chain (SEQ ID NO: 5 or 12).

6. The antibody of claim 3 wherein the antibody comprises all hypervariable region sequences of the YW259.12 antibody heavy chain (SEQ ID NO: 6 or 13).

7. The antibody of any one of claims 3 to 6, further comprising a light chain.

8. The antibody of claim 7 wherein said light chain comprises one, two or three hypervariable sequences of the polypeptide sequence of SEQ ID NO: 15.

9. The antibody of claim 7 wherein said light chain comprises all hypervariable region sequences of the polypeptide sequence of SEQ ID NO: 7 or 15.

10. The antibody of claim 3 selected from the group consisting of antibodies YW259.2, YW259.9, and YW259.12.

11. An isolated anti-PirB/LILRB antibody wherein the full-length IgG form of the antibody specifically binds human PirB (LILRB) with a binding affinity of 5 nM or better.

12. An isolated anti-PirB/LILRB antibody wherein the full-length IgG form of the antibody specifically binds human PirB (LILRB) with a binding affinity of 1 nM or better.

13. The antibody of any one of claims 1-12 that promotes axonal regeneration.

14. The antibody of any one of claims 1-12 that promotes regeneration of CNS neurons.

15. The antibody of any one of claims 1-12 that, at least partially, rescues neurite outgrowth inhibition by Nogo66 and myelin.

16. The antibody of claim any one of claims 1-12, wherein the antibody is a monoclonal antibody.

17. The antibody of any one of claims 1-12, wherein the antibody is selected from the group consisting of a chimeric antibody, a humanized antibody, an affinity matured antibody, a human antibody, and a bispecific antibody.

18. The antibody of any one of claims 1-12, wherein the antibody is an antibody fragment.

19. The antibody of any one of claims 1-12, wherein the antibody is an immunoconjugate.

20. A polynucleotide encoding an antibody of any one of claims 1-12, or a heavy or light chain thereof.

21. A vector comprising the polynucleotide of claim 20.

22. The vector of claim 21, wherein the vector is an expression vector.

23. A host cell comprising a vector of claim 21.

24. The host cell of claim 23, wherein the host cell is prokaryotic.

25. The host cell of claim 23, wherein the host cell is eukaryotic.

26. The host cell of claim 25, wherein the host cell is mammalian.

27. A method for making an anti-PirB/LILRB antibody, said method comprising (a) expressing a vector of claim 22 in a suitable host cell, and (b) recovering the antibody.

28. The method of claim 27, wherein the host cell is prokaryotic.

29. The method of claim 27, wherein the host cell is eukaryotic.

30. A composition comprising an anti-PirB/LILRB antibody of any one of claims 1-12, and a pharmaceutically acceptable excipient.

31. The composition of claim 30, wherein the composition further comprises a second medicament, wherein the anti-PirB/LILRB antibody is a first medicament.

32. The composition of claim 31, wherein the second medicament is a NgR inhibitor.

33. The composition of claim 32 wherein the NgR inhibitor is an anti-NgR antibody.

34. A kit comprising an anti-PirB/LILRB antibody of any one of claims 1-12.

35. A method for promoting axon regeneration comprising administering to a subject in need an effective amount of an anti-PirB/LILRB antibody of any one of claims 1-12.

36. The method of claim 35 wherein the subject is a human patient.

37. The method of claim 36 wherein the survival or neurons is enhanced.

38. The method of claim 36 wherein the outgrowth of neurons is induced.

39. A method of treating a neurodegenerative disease, comprising administering to a subject in need an effective amount of an anti-PirB/LILRB antibody of any one of claims 1-12.

40. The method of claim 39 wherein the neurodegenerative disease is characterized by physical damage to the central nervous system.

41. The method of claim 40 wherein the neurodegenerative disease is brain damage associated with stroke.

42. The method of claim 39 wherein the neurodegenerative disease is selected from the group consisting of trigeminal neuralgia, glossopharyngeal neuralgia, Bell's Palsy, myasthenia gravis, muscular dystrophy, amyotrophic lateral sclerosis (ALS), multiple sclerosis (MS), progressive muscular atrophy, progressive bulbar inherited muscular atrophy, peripheral nerve damage caused by physical injury (e.g., burns, wounds) or disease states such as diabetes, kidney dysfunction or by the toxic effects of chemotherapeutics used to treat cancer and AIDS, herniated, ruptured or prolapsed invertebrate disk syndromes, cervical spondylosis, plexus disorders, thoracic outlet destruction syndromes, peripheral neuropathies such as those caused by lead, dapsone, ticks, prophyria, Gullain-Barre syndrome, Alzheimer's disease, Huntington's Disease, and Parkinson's disease.

43. An anti-idiotype antibody that specifically binds an anti-PirB/LILRB antibody of any one of claims 1-12.


SEQUENCE LISTING (15 sequences)

SEQ ID NO: 1 - protein, 841 aa, Mus sp.
MSCTFTALLRLGLTLSLWIPVLTGSLPKPILRVQPDSVVSRWTKVTFFCEETIGANEYRLYKDGKLYKTVTKNKQKPANKAEFSLSNVDLRNAGQYRCSYSTQYKSSGYSDPLELVVTGDYWTPSLLAQASPVVTSGGYVTLQCESWHNDHKFILTVEGPQKLSWTQDSQYNYSTRKYHALFSVGPVTPNQRWICRCYSYDRNRPYVWSPPSESVELLVSGNLQKPTIKAEPGPVIASKRAMTIWCQGNLDAEVYFLHNEGSQKTQSTQTLQQPGNKGKFFIPSMTRQHAGQYRCYCYGSAGWSQPSDTLELVVTGIYEHYKPRLSVLPSPVVTAGGNMTLHCASDFHYDKFILTKEDKKFGNSLDTEHISSSRQYRALFIIGPTTPTHTGTFRCYGYFKNAPQLWSVPSDLQQILISGLSKKPSLLTHQGHILDPGMTLTLQCYSDINYDRFALHKVGGADIMQHSSQQTDTGFSVANFTLGYVSSSTGGQYRCYGAHNLSSEWSASSEPLDILITGQLPLTPSLSVKPNHTVHSGETVSLLCWSMDSVDTFILSKEGSAQQPLRLKSKSHDQQSQAEFSMSAVTSHLSGTYRCYGAQNSSFYLLSSASAPVELTVSGPIETSTPPPTMSMPLGGLHMYLKALIGVSVAFILFLFILIFILLRRRHRGKFRKDVQKEKDLQLSSGAEEPITRKGELQKRPNPAAATQEESLYASVEDMQTEDGVELNSWTPPEEDPQGETYAQVKPSRLRKAGHVSPSVMSREQLNTEYEQAEEGQGANNQAAESGESQDVTYAQLCSRTLRQGAAASPLSQAGEAPEEPSVYATLAAARPEAVPKDVEQ

SEQ ID NO: 2 - protein, 598 aa, Homo sapiens
MTPIVTVLICLGLSLGPRTHVQTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEDEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAYNLSSEWSAPSDPLDILITGQIHGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGVVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 3 - DNA, 1419 bp, Artificial Sequence
  Description of Artificial Sequence Synthetic polynucleotide
atgggatggtcatgtatcatcctttttctagtagcaactgcaactggagcgtacgctgaggttcagctggtggagtctggcggtggcctggtgcagccagggggctcactccgtttgtcctgtgcagcttctggcttcaccttcactaattcctatattagctgggtgcgtcaggccccgggtaagggcctggaatgggttggtgggatttctccttctggcggttctacttactatgccgatagcgtcaagggccgtttcactataagcgcagacacatccaaaaacacagcctacctacaaatgaacagcttaagagctgaggacactgccgtctattattgtgcaaaatcggtctgggcgttcgcttactggggtcaaggaaccctggtcaccgtctcctcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccctagagtcga

SEQ ID NO: 4 - protein, 465 aa, Artificial Sequence
  Description of Artificial Sequence Synthetic polypeptide
MGWSCIILFLVATATGAYAEVQLVESGGGLVQPGGSLRLSCAASGFTFSNSYISWVRQAPGKGLEWVGGIYPSGGNTNYADSVKGRFTISADTSKNTAYLQMNSLRAEDTAVYYCAKSAWQFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 465 aa, Artificial Sequence
  Description of Artificial Sequence Synthetic polypeptide
MGWSCIILFLVATATGAYAEVQLVESGGGLVQPGGSLRLSCAASGFTFSSNYISWVRQAPGKGLEWVGGINPTGGYTYYAHSVKGRFTISADTSKNTAYLQMNSLRAEDTAVYYCARSVWVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 465 aa, Artificial Sequence
  Description of Artificial Sequence Synthetic polypeptide
MGWSCIILFLVATATGAYAEVQLVESGGGLVQPGGSLRLSCAASGFTFTNSYISWVRQAPGKGLEWVGGISPSGGSTYYADSVKGRFTISADTSKNTAYLQMNSLRAEDTAVYYCAKSVWAFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 233 aa, Artificial Sequence
  Description of Artificial Sequence Synthetic polypeptide
MGWSCIILFLVATATGVHSDIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - DNA, 1419 bp, Artificial Sequence
  Description of Artificial Sequence Synthetic polynucleotide
atgggatggtcatgtatcatcctttttctagtagcaactgcaactggagcgtacgctgaggttcagctggtggagtctggcggtggcctggtgcagccagggggctcactccgtttgtcctgtgcagcttctggcttcaccttcagtaattcctatattagctgggtgcgtcaggccccgggtaagggcctggaatgggttggtgggatttatccttctggcggtaatactaactatgccgatagcgtcaagggccgtttcactataagcgcagacacatccaaaaacacagcctacctacaaatgaacagcttaagagctgaggacactgccgtctattattgtgcaaaaagcgcctggcagttcgcttactggggtcaaggaaccctggtcaccgtctcctcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccctagagtcga

SEQ ID NO: 9 - DNA, 1419 bp, Artificial Sequence
  Description of Artificial Sequence Synthetic polynucleotide
atgggatggtcatgtatcatcctttttctagtagcaactgcaactggagcgtacgctgaggttcagctggtggagtctggcggtggcctggtgcagccagggggctcactccgtttgtcctgtgcagcttctggcttcaccttcagtagtaactatattagctgggtgcgtcaggccccgggtaagggcctggaatgggttggtgggattaatcctactggcggttatacttactatgcccatagcgtcaagggccgtttcactataagcgcagacacatccaaaaacacagcctacctacaaatgaacagcttaagagctgaggacactgccgtctattattgtgcaagaagcgtgtgggtgttcgattactggggtcaaggaaccctggtcaccgtctcctcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccctagagtcga

SEQ ID NO: 10 - protein, 107 aa, Homo sapiens
EVQLVESGGGLVQPGGSLRLSCAASGFTFSSYAMSWVRQAPGKGLEWVSGISGDGGSTYYADSVKGRFTISADTSKNTAYLQMNSLRAEDTAVYYCARGFDYWGQGT

SEQ ID NO: 11 - protein, 110 aa, Artificial Sequence
  Description of Artificial Sequence Synthetic polypeptide
EVQLVESGGGLVQPGGSLRLSCAASGFTFSNSYISWVRQAPGKGLEWVGGIYPSGGNTNYADSVKGRFTISADTSKNTAYLQMNSLRAEDTAVYYCAKSAWQFAYWGQGT

SEQ ID NO: 12 - protein, 109 aa, Artificial Sequence
  Description of Artificial Sequence Synthetic polypeptide
EVQLVESGGGLVQPGGSLRLSCAASGFTFSSNYISWVRQAPGKGLEWVGGINPTGGYTYYAHSVKGRFTISADTSKNTALQMNSLRAEDTAVYYCARSVWVFDYWGQGT

SEQ ID NO: 13 - protein, 110 aa, Artificial Sequence
  Description of Artificial Sequence Synthetic polypeptide
EVQLVESGGGLVQPGGSLRLSCAASGFTFTNSYISWVRQAPGKGLEWVGGISPSGGSTYYADSVKGRFTISADTSKNTAYLQMNSLRAEDTAVYYCAKSVWAFAYWGQGT

SEQ ID NO: 14 - protein, 108 aa, Homo sapiens
DIQMTQSPSSLSASVGDRVTITCRASQSISNYLAWYQQKPGKAPKLLIYAASSLESGVPSRFSGSGSGTDFTLTISSLQPEDFATYYCQQYNSLPWTFGQGTKVEIKR

SEQ ID NO: 15 - protein, 108 aa, Artificial Sequence
  Description of Artificial Sequence Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIKR